(19) United States
(12) Patent Application Publication
(10) Pub. No.: US 2024/0100367 A1
(43) Pub. Date: Mar. 28, 2024

(54) RADIATION THERAPY SYSTEM AND METHOD

(71) Applicant: SHANGHAI UNITED IMAGING HEALTHCARE CO., LTD., Shanghai (CN)

(72) Inventors: Shuguang LIU, Shanghai (CN); Cheng NI, Shanghai (CN); Peng WANG, Shanghai (CN); Jianfeng LIU, Shanghai (CN); Jian ZHANG, Shanghai (CN); Yuelin SHAO, Shanghai (CN)

(73) Assignee: SHANGHAI UNITED IMAGING HEALTHCARE CO., LTD., Shanghai (CN)

(21) Appl. No.: 18/531,741

(22) Filed: Dec. 7, 2023

Related U.S. Application Data
(63) Continuation of application No. PCT/CN2021/138123, filed on Dec. 14, 2021.

(51) Int. Cl. A61N 5/10; A61B 5/055

(52) U.S. Cl. CPC A61N 5/1081; A61B 5/055; A61N 5/1045; A61N 2005/1094; H01J 35/02

(57) ABSTRACT

A radiation therapy system may include a magnetic resonance imaging (MRI) device configured to acquire MRI data with respect to a region of interest (ROI). The MRI device may include a main magnet that is around a longitudinal axis and configured to generate a magnetic field. The MRI device may also include a radiation therapy device configured to perform a treatment on at least one portion of the ROI by delivering, based on the MRI data, therapeutic radiation to the at least one portion of the ROI. The radiation therapy device may be rotatable around the longitudinal axis. The MRI device may also include a first shielding structure configured to provide interference shielding for the MRI device or the radiation therapy device. The radiation therapy device may be rotatable relative to the first shielding structure around the longitudinal axis.

### CROSS-REFERENCE TO RELATED APPLICATIONS

This application is a continuation of International Application No. PCT/CN2021/138123, filed on Dec. 14, 2021, the contents of which are incorporated herein by reference to their entirety.

### TECHNICAL FIELD

The present disclosure generally relates to a radiation therapy system, and more particularly, relates to an image-guided radiation therapy system which combines radiation therapy and magnetic resonance imaging technique.

### BACKGROUND

Radiation therapy on a tumor is currently affected by difficulties to track the variation (e.g., motion) of the tumor in different treatment sessions. Nowadays, various imaging techniques may be applied to provide real-time images of the tumor before or within each treatment session. For example, an image-guided radiation therapy system that combines a magnetic resonance imaging (MRI) device and a radiation therapy device may be used to provide MRI images of the tumor. Due to the combination of the MRI device and the radiation therapy device, there may be interference (e.g., magnetic interference, radiofrequency (RF) interference, microwave interference, radiation interference) between the radiation therapy device (e.g., a radiation source) and the MRI device (e.g., a magnetic body). Therefore, it is desirable to provide a radiation therapy system with a shielding structure disposed between the radiation therapy device and the MRI device.

### SUMMARY

An aspect of the present disclosure provides a radiation therapy system. The radiation therapy system may include a magnetic resonance imaging (MRI) device configured to acquire MRI data with respect to a region of interest (ROI). The MRI device may include a main magnet that is around a longitudinal axis and configured to generate a magnetic field. The MRI device may also include a radiation therapy device configured to perform a treatment on at least one portion of the ROI by delivering, based on the MRI data, therapeutic radiation to the at least one portion of the ROI. The radiation therapy device may be rotatable around the longitudinal axis. The MRI device may also include a first shielding structure configured to provide interference shielding for the MRI device or the radiation therapy device. The radiation therapy device may be rotatable relative to the first shielding structure around the longitudinal axis.

In some embodiments, the first shielding structure may be around the longitudinal axis.

In some embodiments, the radiation therapy device may be at least partially surrounded by the first shielding structure.

In some embodiments, the first shielding structure may include a first opening configured to allow the therapeutic radiation from the radiation therapy device to pass through.

In some embodiments, the radiation therapy device may further include: a radiation source configured to provide the therapeutic radiation; and a gantry configured to support the radiation source. The radiation source may be rotatable with the gantry.

In some embodiments, the radiation therapy device may further include a connection component configured to operably connect the gantry and the first shielding structure. The gantry may be rotatable around the longitudinal axis and supported on the first shielding structure through the connection component.

In some embodiments, the connection component may include one or more bearings.

In some embodiments, the radiation therapy system may further include the one or more second shielding structures mounted on the gantry.

In some embodiments, the one or more second shielding structures may be respectively located at one or more circumferential locations on the gantry.

In some embodiments, the radiation source and the one or more second shielding structures may be evenly distributed on the gantry.

In some embodiments, the gantry may be located within the first shielding structure.

In some embodiments, the gantry may be located outside the first shielding structure.

In some embodiments, there may be a recess at an outer wall of the main magnet. The recess may separate the main magnet into two chambers.

In some embodiments, the radiation therapy device may be at least partially located within the recess.

In some embodiments, the two chambers may be in fluid communication with each other.

In some embodiments, the two chambers may be connected through a neck chamber. The recess may be at least defined by the two chambers and the neck chamber.

In some embodiments, the two chambers may be isolated from each other.

In some embodiments, the radiation therapy device may further include at least one of: a linear accelerator configured to accelerate electrons in an electron beam to produce a radiation beam of the therapeutic radiation, a target configured to receive the accelerated electron beam to produce the radiation beam for the therapeutic radiation, a collimation component configured to collimate the radiation beam of the therapeutic radiation, or a multi-leaf collimator (MLC) configured to make the radiation beam approximate the at least one portion of the ROI.

In some embodiments, at least one of the linear accelerator, the target, the collimation component, or the MLC may be at least partially surrounded by the first shielding structure.

In some embodiments, the first shielding structure may include one or more slots configured to dissipate heat produced by the MRI device or the radiation therapy device, or facilitate cable layout of the radiation therapy system.

In some embodiments, the first shielding structure may be non-rotatable around the longitudinal axis during the treatment.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a block diagram illustrating an exemplary radiation therapy system according to some embodiments of the present disclosure;

FIG. 2 is a flowchart illustrating an exemplary process for applying an therapeutic radiation in a radiation therapy system according to some embodiments of the present disclosure;

FIG. 3 illustrates an exemplary therapeutic device according to some embodiments of the present disclosure;

FIG. 4A shows an upper portion of a cross-sectional view of an exemplary therapeutic device viewed along the X direction according to some embodiments of the present disclosure;

FIG. 4B shows a perspective view of the therapeutic device illustrated in FIG. 4A according to some embodiments of the present disclosure;

FIG. 5A shows an upper portion of a cross-sectional view of an exemplary therapeutic device viewed along the X direction according to some embodiments of the present disclosure;

FIG. 5B shows an upper portion of a cross-sectional view of an exemplary magnetic body of an MRI device viewed along the X direction according to some embodiments of the present disclosure;

FIG. 5C shows an upper portion of a cross-sectional view of an exemplary magnetic body of an MRI device viewed along the X direction according to some embodiments of the present disclosure;

FIG. 6 shows an upper portion of a cross-sectional view of an exemplary therapeutic device 600 viewed along the X direction according to some embodiments of the present disclosure;

FIGS. 7A through 7C show upper portions of cross-sectional views of exemplary configurations between a first shielding structure and a radiation source viewed along the X direction according to some embodiments of the present disclosure;

FIGS. 8A through 9D show an upper portion of a cross-sectional view of configurations of a connection between a gantry and a first shielding structure viewed along the X direction according to some embodiments of the present disclosure;

FIGS. 10A through 10H show a cross-sectional view of exemplary second shielding structures at a circumferential position on a gantry along the radial direction according to some embodiments of the present disclosure;

FIGS. 11A and 11B show a cross-sectional view of exemplary second shielding structures at a circumferential position on a gantry along the radial direction according to some embodiments of the present disclosure;

FIGS. 12A and 12B show cross-sectional views of exemplary therapeutic devices viewed along the Z direction according to some embodiments of the present disclosure;

FIGS. 13A through 14D show a cross-sectional view of exemplary slots along the radial direction according to some embodiments of the present disclosure;

FIG. 15 shows a cross-sectional view of an exemplary therapeutic device viewed along the Z direction according to some embodiments of the present disclosure;

FIG. 16A shows a cross-sectional view of an exemplary therapeutic device viewed along the Z direction according to some embodiments of the present disclosure; and

FIG. 16B shows a cross-sectional view of an exemplary therapeutic device viewed along the Z direction according to some embodiments of the present disclosure.

### DETAILED DESCRIPTION

The following description is presented to enable any person skilled in the art to make and use the present disclosure, and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular exemplary embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of the present disclosure. It is to be expressly understood, however, that the drawings are for the purpose of illustration and description only and are not intended to limit the scope of the present disclosure. It is understood that the drawings are not to scale.

An aspect of the present disclosure provides a radiation therapy system. The system may include a magnetic resonance imaging (MRI) device configured to acquire MRI data with respect to a region of interest (ROI). The MRI device may include a magnetic body (also referred to as main magnet) that is around a longitudinal axis and configured to generate a magnetic field. The system may also include a radiation therapy device configured to perform a treatment on at least one portion of the ROI by applying (or delivering), based on the MRI data, therapeutic radiation to the at least one portion of the ROI. The radiation therapy device may be rotatable around the longitudinal axis. The system may also include a first shielding structure configured to provide interference shielding for the MRI device and/or the radiation therapy device. The first shielding structure may be around the longitudinal axis. The radiation therapy device may be at least partially surrounded by the first shielding structure. The first shielding structure may cover the rotation pathway during the treatment. During the treatment, the radiation therapy device may rotate relative to the first shielding structure around the longitudinal axis. Further, during the treatment, the first shielding structure may be non-rotatable around the longitudinal axis (e.g., keep static relative to the MRI device). At least a portion of the radiation therapy device may rotate around the longitudinal axis within and relative to the first shielding structure during the treatment. The configuration of the radiation therapy system provided in the present disclosure may avoid or reduce an eddy cause by rotation, thereby improving the uniformity of the magnetic field produced by the MRI device under the premise of ensuring the shielding effect.

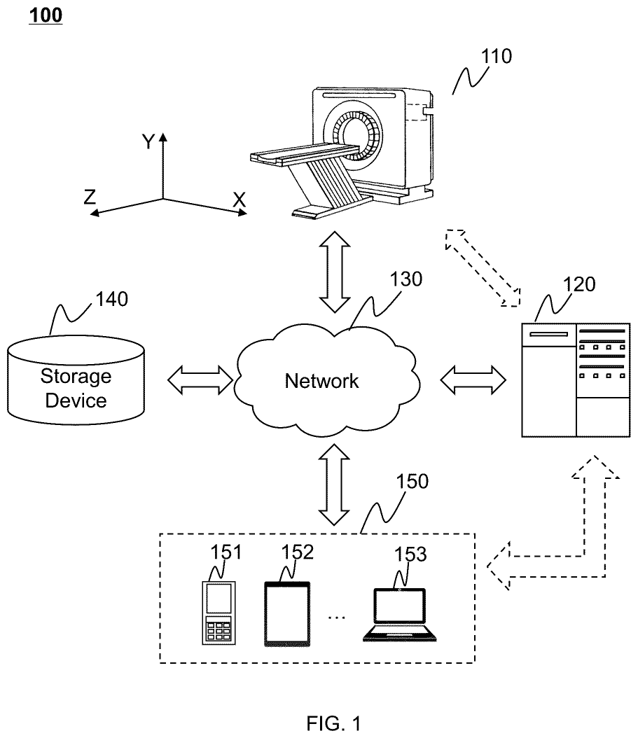

FIG. 1 is a block diagram illustrating an exemplary radiation therapy system 100 according to some embodiments of the present disclosure. In some embodiments, the radiation therapy system 100 may be a multi-modality imaging system including, for example, a positron emission tomography-radiotherapy (PET-RT) system, a magnetic resonance imaging-radiotherapy (MRI-RT) system, etc. For better understanding the present disclosure, an MRI-RT system may be described as an example of the radiation therapy system 100, and not intended to limit the scope of the present disclosure.

As shown in FIG. 1, the radiation therapy system 100 may include a therapeutic device 110, one or more processing engines 120, a network 130, a storage device 140, and one or more terminal devices 150. In some embodiments, the therapeutic device 110, the one or more processing engines 120, the storage device 140, and/or the terminal device 150 may be connected to and/or communicate with each other via a wireless connection (e.g., the wireless connection provided by the network 130), a wired connection (e.g., the wired connection provided by the network 130), or any combination thereof.

The therapeutic device 110 may include a magnetic resonance imaging (MRI) component (hereinafter referred to as “MRI device”). The MRI device may generate image data associated with magnetic resonance (MR) signals via scanning a subject or a part of the subject (e.g., a region of interest (ROI) of the subject). In some embodiments, the subject may include a body, a substance, an object, or the like, or any combination thereof. In some embodiments, the subject may include a specific portion of a body, a specific organ, or a specific tissue, such as head, brain, neck, body, shoulder, arm, thorax, cardiac, stomach, blood vessel, soft tissue, knee, feet, or the like, or any combination thereof. In some embodiments, the therapeutic device 110 may transmit the image data via the network 130 to the one or more processing engines 120, the storage device 140, and/or the terminal device 150 for further processing. For example, the image data may be sent to the one or more processing engines 120 for generating an MRI image, or may be stored in the storage device 140.

The therapeutic device 110 may also include a radiation therapy component (hereinafter referred to as “radiation therapy device”). The radiation therapy device may provide radiation for target region (e.g., a tumor) treatment. For example, the radiation therapy device may provide radiation for a target region (e.g., a tumor) of an ROI of a subject (e.g., a patient). The radiation used herein may include a particle ray, a photon ray, etc. The particle ray may include neutron, proton, electron, p-meson, heavy ion, a-ray, or the like, or any combination thereof. The photon ray may include X-ray, y-ray, ultraviolet, laser, or the like, or any combination thereof. For illustration purposes, a radiation therapy device associated with X-ray may be described as an example. In some embodiments, the therapeutic device 110 may generate a certain dose of X-rays to perform radiotherapy under the assistance of the image data provided by the MRI device. For example, the image data may be processed to locate a tumor and/or determine the dose of X-rays.

The one or more processing engines 120 may process data and/or information obtained from the therapeutic device 110, the storage device 140, and/or the terminal device 150. For example, the one or more processing engines 120 may process image data and reconstruct at least one MRI image based on the image data. As another example, the one or more processing engines 120 may determine the position of the treatment region and the dose of radiation based on the at least one MRI image. The MRI image may provide advantages including, for example, superior soft-tissue contrast, high resolution, geometric accuracy, which may allow accurate positioning of the treatment region. The MRI image may be used to detect the variance of the treatment region (e.g., a tumor regression or metastasis) during the time when the treatment plan is determined and the time when the treatment is carried out, such that an original treatment plan may be adjusted accordingly. The original treatment plan may be determined before the treatment commences. For instance, the original treatment plan may be determined at least one day, or three days, or a week, or two weeks, or a month, etc., before the treatment commences.

In the original or adjusted treatment plan, the dose of radiation may be determined according to, for example, synthetic electron density information. In some embodiments, the synthetic electron density information may be generated based on the MRI image.

In the present disclosure, the X axis, the Y axis, and the Z axis shown in FIG. 1 may form an orthogonal coordinate system. The X axis and the Z axis shown in FIG. 1 may be horizontal, and the Y axis may be vertical. As illustrated, the positive X direction along the X axis may be from the left side to the right side of the therapeutic device 110 seen from the direction facing the front of the therapeutic device 110; the positive Y direction along the Y axis shown in FIG. 1 may be from the lower part to the upper part of the therapeutic device 110; the positive Z direction along the Z axis shown in FIG. 1 may refer to a direction in which the object is moved out of the scanning channel (or referred to as the bore) of the therapeutic device 110.

In some embodiments, the one or more processing engines 120 may be a single processing engine that communicates with and process data from the MRI device and the radiation therapy device of the therapeutic device 110. Alternatively, the one or more processing engines 120 may include at least two processing engines. One of the at least two processing engines may communicate with and process data from the MRI device of the therapeutic device 110, and another one of the at least two processing engines may communicate with and process data from the radiation therapy device of the therapeutic device 110. In some embodiments, the one or more processing engines 120 may include a treatment planning system. The at least two processing engines may communicate with each other.

In some embodiments, the one or more processing engines 120 may be a single server or a server group. The server group may be centralized or distributed. In some embodiments, the one or more processing engines 120 may be local to or remote from the therapeutic device 110. For example, the one or more processing engines 120 may access information and/or data from the therapeutic device 110, the storage device 140, and/or the terminal device 150 via the network 130. As another example, the one or more processing engines 120 may be directly connected to the therapeutic device 110, the terminal device 150, and/or the storage device 140 to access information and/or data. In some embodiments, the one or more processing engines 120 may be implemented on a cloud platform. The cloud platform may include a private cloud, a public cloud, a hybrid cloud, a community cloud, a distributed cloud, an inter-cloud, a multi-cloud, or the like, or any combination thereof.

The network 130 may include any suitable network that can facilitate the exchange of information and/or data for the radiation therapy system 100. In some embodiments, one or more components of the radiation therapy system 100 (e.g., the therapeutic device 110, the one or more processing engines 120, the storage device 140, or the terminal device 150) may communicate information and/or data with one or more other components of the radiation therapy system 100 via the network 130. For example, the one or more processing engines 120 may obtain image data from the therapeutic device 110 via the network 130. As another example, the one or more processing engines 120 may obtain user instructions from the terminal device 150 via the network 130. The network 130 may include a public network (e.g., the Internet), a private network (e.g., a local area network (LAN), a wide area network (WAN)), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, or the like, or any combination thereof. In some embodiments, the network 130 may include one or more network access points. For example, the network 130 may include wired and/or wireless network access points such as base stations and/or internet exchange points through which one or more components of the radiation therapy system 100 may be connected to the network 130 to exchange data and/or information.

The storage device 140 may store data, instructions, and/or any other information. In some embodiments, the storage device 140 may store data obtained from the one or more processing engines 120 and/or the terminal device 150. In some embodiments, the storage device 140 may store data and/or instructions that the one or more processing engines 120 may execute or use to perform exemplary methods described in the present disclosure. In some embodiments, the storage device 140 may include a mass storage device, a removable storage device, a cloud based storage device, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), a digital versatile disk ROM, etc. In some embodiments, the storage device 140 may be implemented on a cloud platform as described elsewhere in the present disclosure.

In some embodiments, the storage device 140 may be connected to the network 130 to communicate with one or more other components of the radiation therapy system 100 (e.g., the one or more processing engines 120 or the terminal device 150). One or more components of the radiation therapy system 100 may access the data or instructions stored in the storage device 140 via the network 130. In some embodiments, the storage device 140 may be part of the one or more processing engines 120.

The terminal device 150 may be connected to and/or communicate with the therapeutic device 110, the one or more processing engines 120, and/or the storage device 140. For example, the one or more processing engines 120 may acquire a scanning protocol from the terminal device 150. As another example, the terminal device 150 may obtain image data from the therapeutic device 110 and/or the storage device 140. In some embodiments, the terminal device 150 may include a mobile device 151, a tablet computer 152, a laptop computer 153, or the like, or any combination thereof. For example, the mobile device 151 may include a mobile phone, a personal digital assistance (PDA), a gaming device, a navigation device, a point of sale (POS) device, a laptop, a tablet computer, a desktop, or the like, or any combination thereof. In some embodiments, the terminal device 150 may include an input device, an output device, etc. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to the one or more processing engines 120 via, for example, a bus, for further processing. Other types of the input device may include a cursor control device, such as a mouse, a trackball, or cursor direction keys, etc. The output device may include a display, a speaker, a printer, or the like, or any combination thereof. In some embodiments, the terminal device 150 may be part of the one or more processing engines 120.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. For example, the storage device 140 may be a data storage including cloud computing platforms, such as public cloud, private cloud, community, hybrid clouds, etc. In some embodiments, the one or more processing engines 120 may be integrated into the therapeutic device 110. However, those variations and modifications do not depart the scope of the present disclosure.

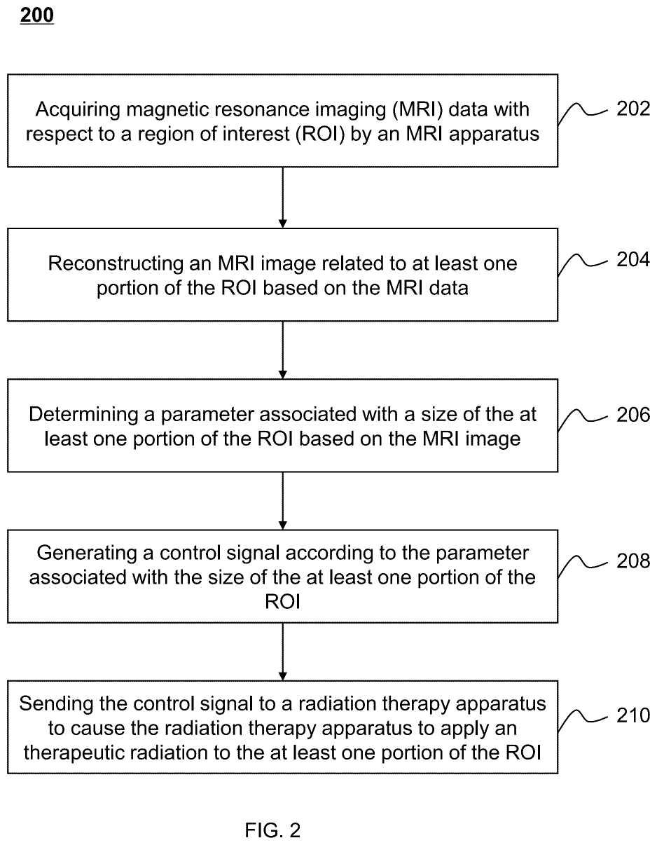

FIG. 2 is a flowchart of an exemplary process 200 for applying a therapeutic radiation by a radiation therapy system according to some embodiments of the present disclosure. In some embodiments, one or more operations of the process 200 illustrated in FIG. 2 may be implemented in the radiation therapy system 100 illustrated in FIG. 1. For example, the process 200 illustrated in FIG. 2 may be stored in the storage device 140 in the form of instructions, and invoked and/or executed by the one or more processing engines 120 illustrated in FIG. 1. For illustration purposes, the implement of the process 200 in the one or more processing engines 120 is described herein as an example. It shall be noted that the process 200 can also be similarly implemented in the terminal device 150.

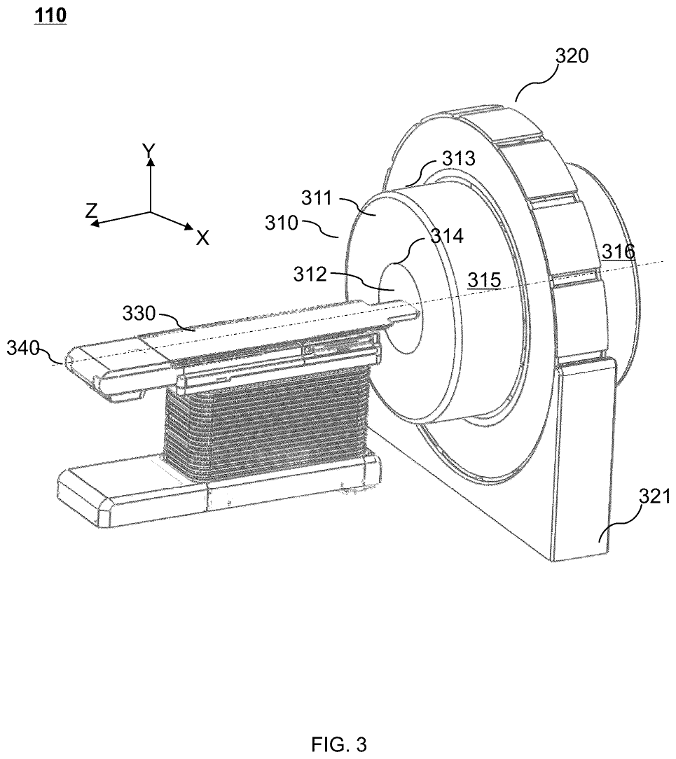

In 202, the one or more processing engines 120 may acquire magnetic resonance imaging (MRI) data (also referred to as image data) with respect to a region of interest (ROI) by an MRI device. The MRI data may be MR signals received by an RF coil from a subject. More detailed description related to the MR signals may be found elsewhere in the present disclosure at, for example, FIG. 3 and the description thereof.

In some embodiments, an ROI may refer to a treatment region associated with a tumor. The treatment region may be a region of a subject (e.g., a body, a substance, an object). In some embodiments, the ROI may be a specific portion of a body, a specific organ, or a specific tissue, such as head, brain, neck, body, shoulder, arm, thorax, cardiac, stomach, blood vessel, soft tissue, knee, feet, or the like, or any combination thereof.

In 204, the one or more processing engines 120 may reconstruct an MRI image related to at least one portion of the ROI based on the MRI data. The MRI image may be reconstructed as a distribution of atomic nuclei inside the subject based on the MRI data. Different kinds of imaging reconstruction techniques for the image reconstruction procedure may be employed. Exemplary image reconstruction techniques may include Fourier reconstruction, constrained image reconstruction, regularized image reconstruction in parallel MRI, or the like, or a variation thereof, or any combination thereof.

The MRI image may be used to determine a therapeutic radiation to a tumor. For example, the one or more processing engines 120 may determine the position of the tumor and the dose of radiation according to the MRI image. In some embodiments, it may take at least several minutes to reconstruct an MRI image representing a large imaging region. In some embodiments, in order to generate the MRI image during a relative short time period (e.g., every second), the one or more processing engines 120 may reconstruct an initial image representing a smaller imaging region (e.g., at least one portion of the ROI) compared to that of the MRI image representing a large imaging region, and then combine the initial image with the MRI image representing a large imaging region. For example, the one or more processing engines 120 may replace a portion of the MRI image representing a large imaging region related to the ROI with the initial image. The MRI image representing a large imaging region may include information of non-ROI (e.g., a healthy tissue) near the ROI and that of the ROI. In some embodiments, the MRI image representing a large imaging region may be acquired and reconstructed before the therapeutic radiation on the tumor. For example, the MRI image representing a large imaging region may be acquired less than 1 day, or half a day, or 6 hours, or 3 hours, or 1 hour, or 45 minutes, or 30 minutes, or 20 minutes, or 15 minutes, or 10 minutes, or 5 minutes, etc., before the radiation source starts emitting a radiation beam for treatment. In some embodiments, the MRI image representing a large imaging region may be obtained from a storage device in the radiation therapy system 100, such as the storage device 140.

In 206, the one or more processing engines 120 may determine a parameter associated with a size of the at least one portion of the ROI based on the MRI image. In some embodiments, the parameter associated with a size of the at least one portion of the ROI may include the size of the cross section of a tumor which has the maximum area and is perpendicular to the direction of the radiation beams impinging on the at least one portion of the ROI. In some embodiments, the parameter associated with a size of the at least one portion of the ROI may indicate the shape of the cross section of the tumor. For example, the parameter associated with a size of at least one portion of the ROI may indicate that the shape of the cross section of the tumor is circle, and further indicate the diameter of the circle. In some embodiments, to determine the parameter associated with a size of at least one portion of the ROI, the one or more processing engines 120 may extract texture information from the MRI image, and determine texture features that are indicative of the ROI by identifying frequent texture patterns of the ROI in the extracted texture information. Then, the one or more processing engines 120 may measure the size of the region which includes the texture features in the MRI image, and determine the parameter associated with the size of the ROI.

In 208, the one or more processing engines 120 may generate a control signal according to the parameter associated with the size of at least one portion of the ROI. The control signal may be dynamically adjusted based on the plurality of MRI images taken at different time points. In some embodiments, the control signal may include parameters associated with the therapeutic radiation on the tumor. For example, the control signal may include the dosage of X-rays and a duration of the radiation beam. For another example, the control signal may include parameters of multi-leaf collimator (MLC) that determines the shape of the radiation beam projected on the subject. The MLC may include a plurality of individual leaves of high atomic numbered materials (e.g., tungsten) moving independently in and out of the path of the radiation beam. In some embodiments, the control signal may include parameters associated with movements of one or more components of a radiation therapy device. For example, the control signal may include a parameter associated with one or more positions of a radiation source of the radiation therapy device (e.g., the radiation therapy device in the therapeutic device 110, a radiation therapy device 320). For another example, the control signal may include a parameter associated with a height or a position of a platform of the radiation therapy apparatus (e.g., a location of the platform 308 of the treatment table 330 along an axis of the magnetic body 311) to properly position a patient so that the treatment region (e.g., a cancerous tumor or lesion) in the patient may properly receive the radiation beam from the radiation therapy device.

In 210, the one or more processing engines 120 may send the control signal to a radiation therapy device to cause the radiation therapy device to apply the therapeutic radiation. During the therapeutic radiation, the radiation source of the radiation therapy device may rotate, and the dosage of X-rays, duration of radiation beam from a radiation source, the shape of MLC and the position of the platform may be varied. In some embodiments, the radiation beam may be emitted only when the radiation source of the radiation therapy device rotates to certain angles (e.g., 60 degrees, 120 degrees, 180 degrees, 240 degrees, 300 degrees, 360 degrees). For example, an intensity modulated radiation therapy (IMRT) may be applied. The radiation source may stop rotating intermittently. The radiation source may rotate to a desired position, pause there, and emit a radiation beam, and then resume to rotate. In some embodiments, the radiation source may rotate continuously, and emit a radiation beam continuously or intermittently. In some embodiments, the radiation source may continuously emit the radiation beam while rotating.

In some embodiments, as described above, a treatment region (e.g., a region including a tumor) may be determined according to the image data acquired from the MRI device. Then a radiation beam may be generated by a radiation source of the radiation therapy device to perform the therapeutic radiation to the treatment region. For example, the dosage of the radiation beam and/or the position of the treatment region may be determined in real-time with the assistance of the MRI device.

It should be noted that the above description is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, operations 202 and 204 may be performed simultaneously.

FIG. 3 illustrates an exemplary therapeutic device 110 according to some embodiments of the present disclosure. As shown in FIG. 3, the X axis, the Y axis, and the Z axis may correspond to those in FIG. 1. As illustrated in FIG. 3, the therapeutic device 110 may include an MRI device 310, a radiation therapy device 320, and a treatment table 330. In some embodiments, the MRI device 310 may generate the MRI data as described in connection with operation 202, and the radiation therapy device 320 may apply the therapeutic radiation as described in connection with operation 210.

The MRI device 310 may include a bore 312, a magnetic body 311, one or more gradient coils (not shown), and one or more radiofrequency (RF) coils (not shown). The MRI device 310 may be configured to acquire image data from an imaging region. For example, the image data may relate to the treatment region associated with a tumor. In some embodiments, the MRI device 310 may be a permanent magnet MRI scanner, a superconducting electromagnet MRI scanner, or a resistive electromagnet MRI scanner, etc., according to the types of the magnetic body 311. In some embodiments, the MRI device 310 may be a high-field MRI scanner, a mid-field MRI scanner, and a low-field MRI scanner, etc., according to the intensity of the magnetic field. In some embodiments, the MRI device 310 may be of a closed-bore (cylindrical) type, an open-bore type, or the like.

The magnetic body 311 may be around a longitudinal axis 340 of the bore 312 and generate a static magnetic field B0. For example, the magnetic body 311 may include an annulus structure around the longitudinal axis 340 with the bore 312 passing through the magnetic body 311 along the extending direction (i.g., the Z direction) of the longitudinal axis 340. The magnetic body 311 may be of various types including, for example, a permanent magnet, a superconducting electromagnet, a resistive electromagnet, etc. The superconducting electromagnet may include niobium, vanadium, technetium alloy, etc.

In some embodiments, the magnetic body 311 may include a plurality of coils disposed in the magnetic body 311 and configured to generate a magnetic field. The plurality of coils may be arranged coaxially around the longitudinal axis 340.

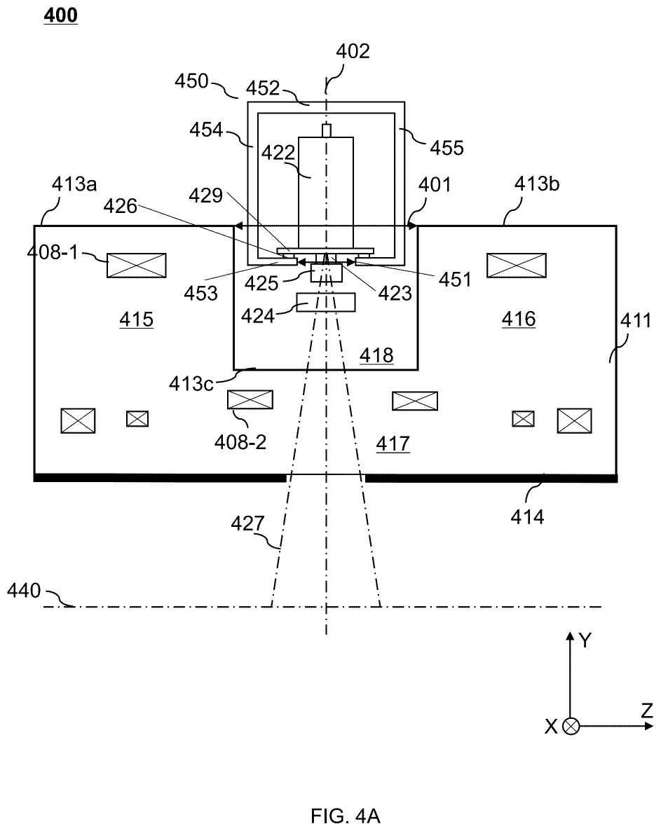

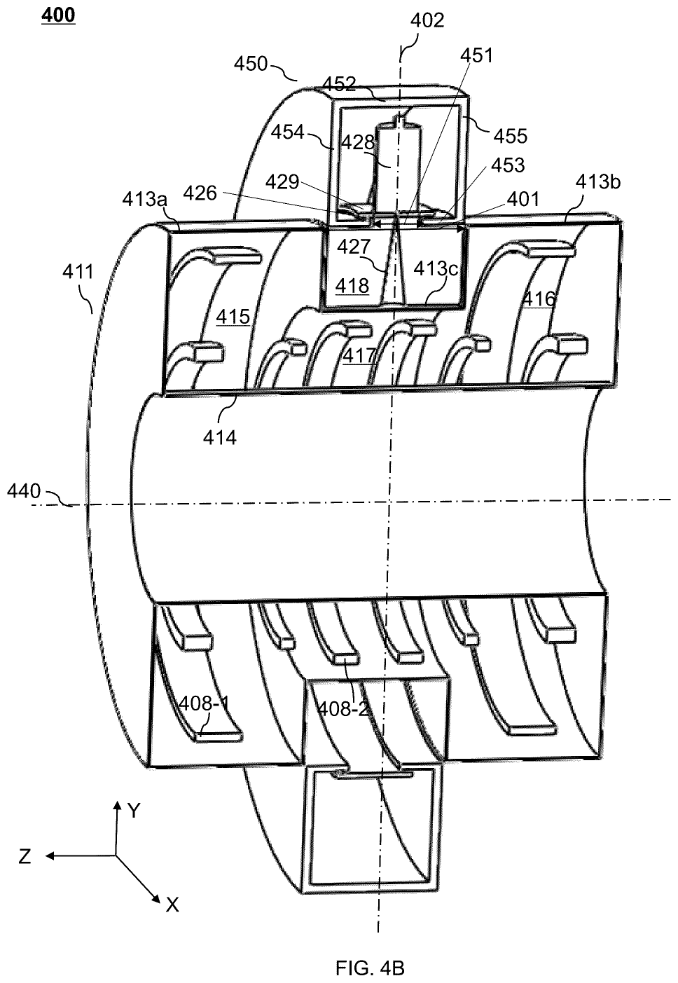

Merely by way of example, the magnetic body 311 may include a plurality of superconducting coils including a plurality of main magnetic coils (e.g., 408-2 shown in FIG. 4A) and a plurality of shielding magnetic coils (e.g., 408-1 shown in FIG. 4A), and a cryostat.

The plurality of main magnetic coils and the plurality of shielding magnetic coils may be accommodated in the cryostat and maintained in the superconductive state under a certain condition (e.g., when both the coils are immerged in a cooling medium in the cryostat).

The cryostat may have a shape of an annulus with the longitudinal axis 340. The plurality of main magnetic coils may be arranged coaxially along the longitudinal axis 340 to generate a uniform magnetic field (e.g., a static magnetic field B0) within a specific region (e.g., a region within the bore 312 of the therapeutic device 110) when the plurality of main magnetic coils carry an electric current along a first direction.

The plurality of shielding magnetic coils may also be arranged coaxially along the longitudinal axis 340 at a larger radius from the longitudinal axis 340 than the plurality of main magnetic coils. The plurality of shielding magnetic coils may carry an electric current along a second direction that is opposed to the first direction. The plurality of shielding magnetic coils may help shield the magnetic field generated by the plurality of main magnetic coils on a region outside the MRI device of the therapeutic device 110.

The radiation therapy device 320 may include a gantry, a radiation source, and a pedestal 321. The radiation source may be configured to emit a radiation beam towards the treatment region in the bore 312. The radiation beam may have a central axis perpendicular to the longitudinal axis 340. The radiation beam may be an X-ray beam, an electron beam, a gamma ray source, a proton ray source, etc. The gantry may be configured to support the radiation source. The gantry, together with the radiation source mounted thereon, may be able to rotate around the longitudinal axis 340 of the bore 312 and/or a point called the isocenter of the bore 312. Merely by way of example, the gantry, together with the radiation source mounted thereon, may be able to rotate any angle, e.g., 90 degrees, 180 degrees, 360 degrees, 450 degrees, 540 degrees, around the longitudinal axis 340. The gantry and the radiation source may be further supported by the pedestal 321.

In some embodiments, the radiation source may include a linear accelerator, a target, a collimation component, and a multi-leaf collimator (MLC).

The linear accelerator may be configured to accelerate charged subatomic particles or ions to a high speed. In some embodiments, the linear accelerator may accelerate electrons using microwave technology. For example, the linear accelerator may accelerate electrons in an electron beam with energy group between 4 MeV to 22 MeV using high radiofrequency (RF) electromagnetic waves. The linear accelerator may be mounted to the gantry that is capable of rotating around the longitudinal axis 340 and may enable the radiation beam to be emitted from an arbitrary circumferential position.

The target may be configured to receive the accelerated charged subatomic particles or ions (e.g., an electron beam) from the linear accelerator to produce the radiation beam for the therapeutic radiation. For example, the electron beam may collide with the target to generate high-energy X-rays according to the bremsstrahlung effect. In some embodiments, the target may be located near the exit window of the linear accelerator to receive the accelerated electron beam. In some embodiments, the target may be made of materials including aluminium, copper, silver, tungsten, or the like, or any combination thereof. Alternatively, the target may be made of composite materials including tungsten and copper, tungsten and silver, tungsten and aluminium, or the like, or any combination thereof.

The radiation beam from the target may pass through the collimation component to form a beam with a specific shape (e.g., a cone beam). In some embodiments, the collimation component may include a primary collimator, a flattening filter and at least one secondary collimator.

The MLC may be configured to reshape the radiation beam. For example, the MLC may adjust the irradiating shape, the irradiating area, etc., of the radiation beam. The MLC may make the radiation beam approximate the treatment area (e.g., a tumor) of a subject (e.g., a patient). The MLC may stay fixed relative to the linear accelerator, thus rotating together with the linear accelerator around the longitudinal axis 340. The MLC may include a plurality of individual leaves of high atomic numbered materials (e.g., tungsten) moving independently in and out of the path of the radiation beam in order to block it. The shape of the radiation beam may vary when the plurality of individual leaves move in and out, forming different slots that simulates the cross section of the tumor viewed from a central axis of the radiation beam (i.e., the vertical dotted line 402 shown in FIG. 4A).

The radiation source may generate the radiation beam according to one or more parameters. Exemplary parameter may include a parameter of the radiation beam, a parameter of the radiation source, or a parameter of the treatment table 330. For example, the parameter of the radiation beam may include an irradiating intensity, an irradiating angle, an irradiating distance, an irradiating area, an irradiating time, an intensity distribution, or the like, or any combination thereof. The parameter of the radiation source may include a position, a rotating angle, a rotating speed, a rotating direction, the configuration of the radiation source, or the like, or any combination thereof. In some embodiments, the generation of the radiation beam by the radiation source may take into consideration energy loss of the radiation beam due to, e.g., the magnetic body 311 located in the pathway of the radiation beam that may absorb at least a portion of the radiation beam. For example, the irradiating intensity of the radiation beam may be set larger than that in the situation in which there is no energy loss due to, e.g., the absorption by the magnetic body 311 accordingly to compensate the energy loss such that the radiation beam of a specific intensity may impinge on a treatment region (e.g., a tumor).

The treatment table 330 may be configured to support a subject (e.g., a patient) and carry the subject in or out of the bore 312. In some embodiments, the treatment table 330 may move along the Z direction and get into or out of the bore 312. In some embodiments, the treatment table 330 may move two-dimensionally, three-dimensionally, four-dimensionally, five-dimensionally or six-dimensionally. In some embodiments, the treatment table 330 may move according to the variance (e.g., position change) of the tumor estimated by, for example, a real-time MRI image obtained during a treatment.

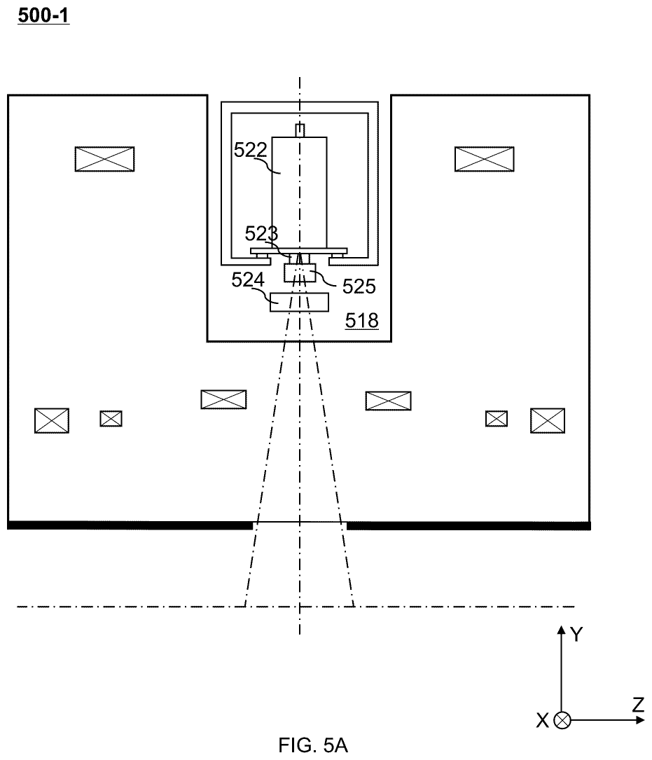

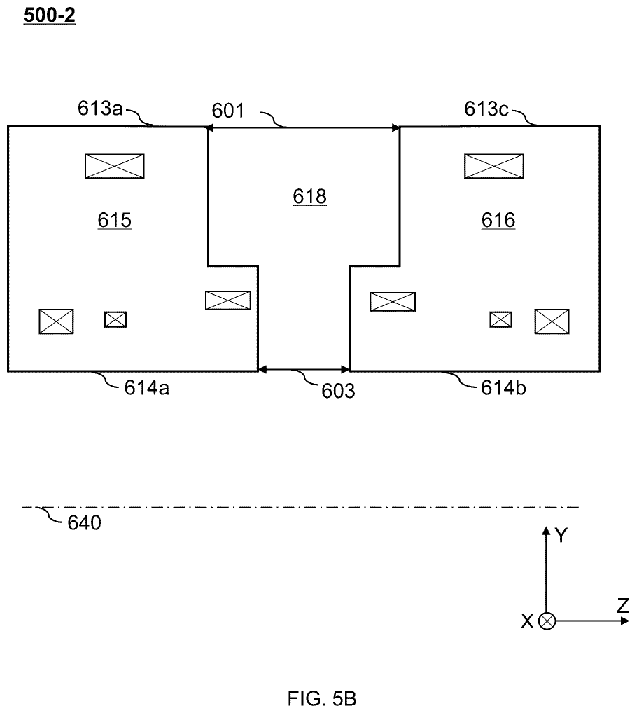

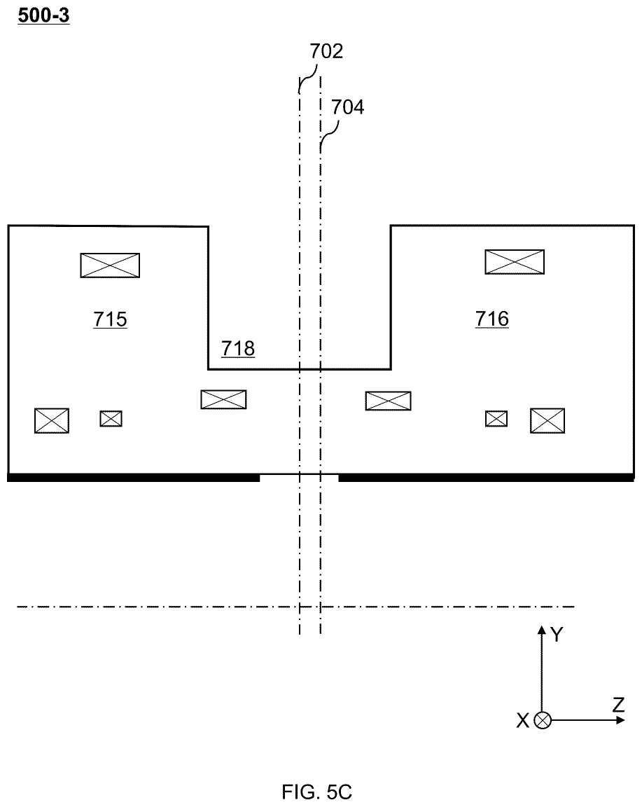

In some embodiments, the magnetic body 311 may further include a recess (not shown) at its outer wall 313. The recess may be disposed around the entire circumference of the magnetic body 311. For example, the recess may have a shape of an annulus surrounding the magnetic body 311. In some embodiments, the recess may be disposed around part of the circumference of the magnetic body 311. For example, the recess may have a shape of one or more arcs around the magnetic body 311. In some embodiments, at least a portion of the radiation source of the radiation therapy device 320 may be located in the recess (e.g., as shown in FIG. 4A and FIG. 5A). In some embodiments, the radiation source may move along at least a portion of an entire path of rotation within the recess.

In some embodiments, the recess may separate the magnetic body 311 into a first chamber 315 and a second chamber 316. In some embodiments, the magnetic body 311 may include at least one outer wall 313 and at least one inner wall 314 coaxially around the longitudinal axis 340. The at least one inner wall 314 may be located closer to the longitudinal axis 340 than the at least one outer wall 313. The bore 312 may be defined by the at least one inner wall 314. The recess may be between the at least one outer wall 313 and the at least one inner wall 314. The recess may have an opening formed at the at least one outer wall 313. In some embodiments, the first chamber 315 and the second chamber 316 may be in fluid communication with (e.g., as shown in FIG. 4A) or isolated from each other (e.g., as shown in FIG. 5B).

In some embodiments, the recess may be coaxial with the magnetic body 311 along the Y direction (e.g., as shown in FIG. 4A). Alternatively, the recess may not be coaxial with the magnetic body 311 along the Y direction (e.g., as shown in FIG. 5C). More details regarding the recess may be found elsewhere in the present disclosure (e.g., the description in connection with FIGS. 4A, 5B, and 5C).

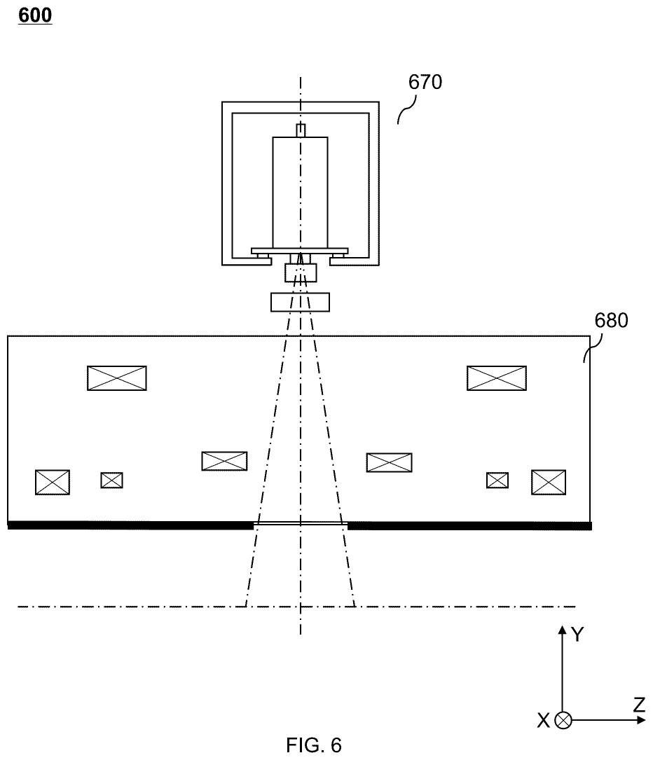

In some embodiments, there may be no recess at the outer wall 313. The radiation therapy device 320 may be disposed around the outer circumference of the magnetic body 311 (as shown in FIG. 3 and FIG. 6).

In some embodiments, the radiation therapy device 320 may further include a first shielding structure configured to provide shielding for interference (e.g., magnetic interference, radiofrequency (RF) interference, microwave interference, radiation interference) between the radiation therapy device 320 (e.g., the radiation source) and the MRI device 310 (e.g., the magnetic body 311). During the treatment, the radiation therapy device may rotate relative to the first shielding structure around the longitudinal axis. Further, during the treatment, the first shielding structure may be non-rotatable around the longitudinal axis (e.g., keep static relative to the MRI device). The first shielding structure may be around the longitudinal axis 340. For example, the first shielding structure may have a shape of an annulus around the longitudinal axis 340. In some embodiments, the first shielding structure may cover the rotation pathway of the radiation source. For example, the first shielding structure may have a shape of an annulus around the entire circumference of the magnetic body 311. As another example, if the radiation source is able to rotatable around the longitudinal axis 340 for a rotation range less than 360 degrees, the first shielding structure may have a shape of one or more arcs around part of the circumference of the magnetic body 311 corresponding to the rotation range of the radiation source. In some embodiments, the radiation therapy device 320 may be at least partially surrounded by the first shielding structure. Specifically, at least one of the linear accelerator, the target, the collimation component, or the MLC may be at least partially surrounded by the first shielding structure (as shown in FIGS. 4A through 7C). In this way, the first shielding structure may form a shielding channel. During the treatment, at least a portion of the radiation source may rotate around the longitudinal axis 340 within and relative to the shielding channel, which may avoid or reduce an eddy cause by rotation, thereby improving the uniformity of the magnetic field produced by the MRI device 310 under the premise of ensuring the shielding effect.

In some embodiments, before the treatment, the location of the first shielding structure may be adjusted to cover the rotation pathway of the radiation therapy device 320 during the treatment. For example, the first shielding structure may be a semi-ring structure around the longitudinal axis 340. For a treatment, the radiation source of the radiation therapy device 320 is required to rotate within a rotation range of 180°-360°. Assuming that before the treatment, the first shielding structure is located at an initial position that covers a rotation range of 0°-180°, the first shielding structure may be adjusted from the initial position to a target position that covers the rotation range of 180°-360°. During the treatment, the first shielding structure may be non-rotatable around the longitudinal axis 340 and fixed at the target position to cover the rotation pathway of the radiation source of the radiation therapy device 320.

In some embodiments, the first shielding structure may include a first opening configured to allow the therapeutic radiation from the radiation therapy device 320 to pass through, so that the therapeutic radiation is able to emit toward the longitudinal axis 340 (e.g., a target region to be treated of a patient in the bore 312). In some embodiments, the cross-section of the first shielding structure along the radial direction may have any shape, such as a circle, a rectangle, a square, etc.

In some embodiments, the first shielding structure may include a plurality of shielding layers. At least one of the plurality of shielding layers may be used to reduce magnetic interference between one or more components of the MRI device 310 and the radiation therapy device 320. For example, the first shielding structure may include a magnetic shielding layer configured to shield the magnetic field produced by the MRI device 310 (e.g., the main magnetic coils, the shielding magnetic coils, the gradient coils) in case that the electrons may be influenced by the magnetic field.

Additionally, at least one of the plurality of shielding layers may be used to reduce the RF and/or microwave interference between one or more components of the MRI device 310 and the radiation therapy device 320. For example, the first shielding structure may include an electromagnetic shielding layer configured to shield the RF signals produced by the MRI device 310 (e.g., the RF coils) and the microwave produced by the radiation therapy device 320.

The plurality of shielding layers may be made of same material and/or different materials. For example, both the electromagnetic shielding layer and the magnetic shielding layer may be made of high magnetic susceptibility and permeability material (e.g., non-oriented silicon steel), or one of the electromagnetic shielding layer and the magnetic shielding layer is made of high electric conductivity and magnetic permeability material. In some embodiments, the plurality of shielding layers may be magnetically and/or electrically isolated from each other with a suitable dielectric material, such as air or plastic, between them.

Additionally or alternatively, at least one of the plurality of shielding layers may be used to protect one or more components of the MRI device 310 from the radiation produced by the radiation therapy device 320. For example, one shielding layer of the plurality of shielding layers may be made of a material that is able to absorb the radiation produced by the radiation beam of the radiation therapy device 320. Exemplary material that is able to absorb the radiation may include materials for absorbing photon ray and/or materials for absorbing neutron ray. The materials for absorbing photon ray may include steel, aluminum, lead, tungsten, etc. The materials for absorbing neutron ray may include boron, graphite, etc. It should be noted that, in some embodiments, the first shielding structure may be made only with radiation absorbing material, without high magnetic susceptibility and permeability material. In this way, the first shielding structure may only provide radiation shielding for one or more components of the MRI device 310.

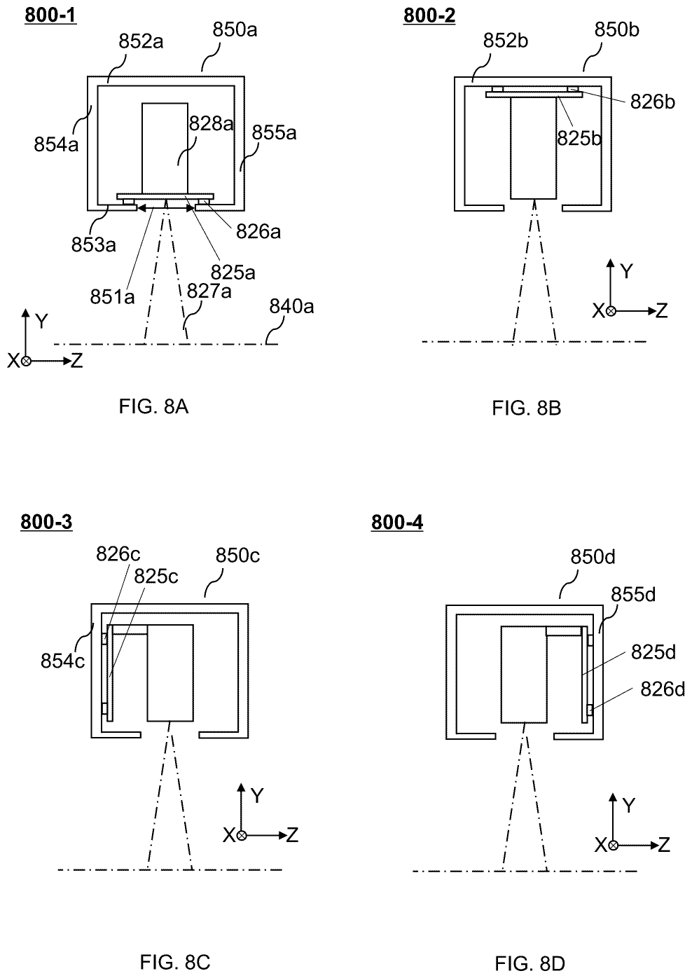

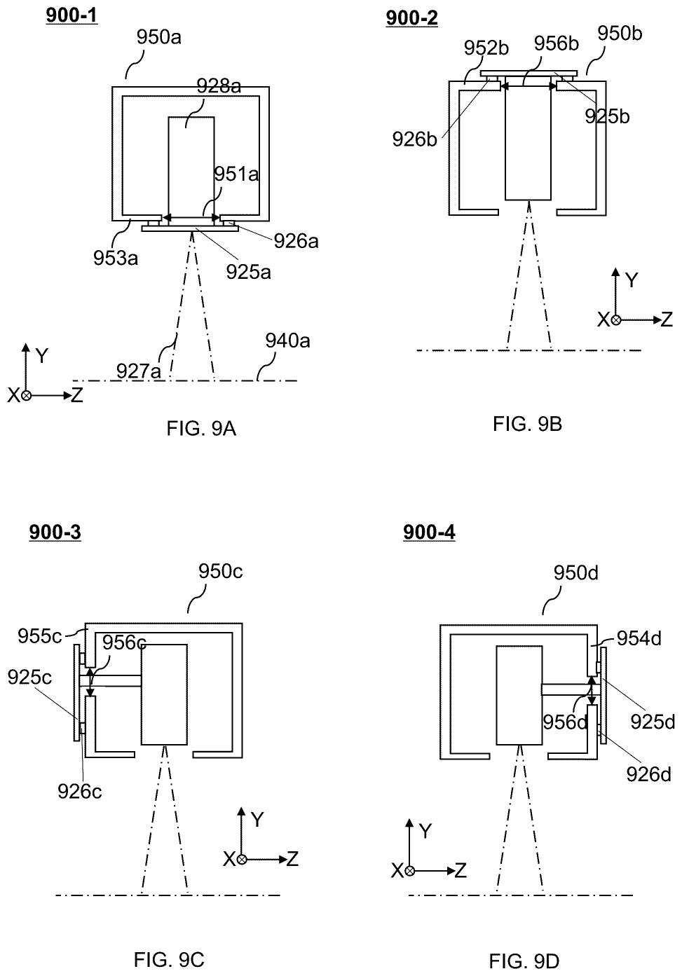

In some embodiments, the radiation therapy device 320 may further include a connection component configured to operably connect the gantry and the first shielding structure. The gantry may be rotatable around the longitudinal axis 340 relative to the first shielding structure through the connection component during the treatment. In some embodiments, the connection component may include one or more bearings. Details regarding the configuration of the connection between the gantry and the first shielding structure may be found elsewhere in the present disclosure (e.g., the description in connection with FIGS. 8A through 9D).

In some embodiments, the first shielding structure may be mounted on the pedestal 321. For example, the first shielding structure may be fixed with the pedestal 321. As another example, the first shielding structure may be rotatable relative to the pedestal 321.

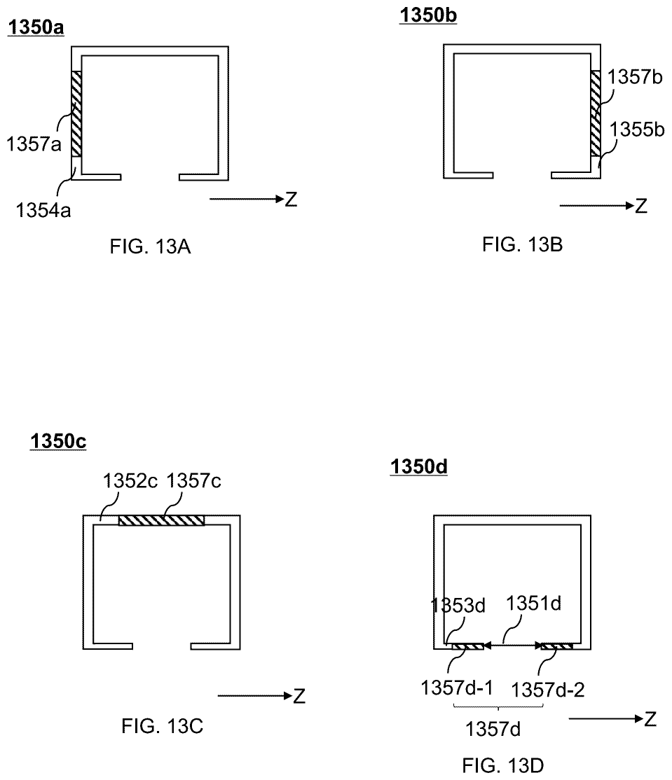

In some embodiments, the first shielding structure may include one or more slots configured to dissipate heat produced by the MRI device 310 and/or the radiation therapy device 320, and/or facilitate cable layout of the radiation therapy device 110. Details regarding the configuration of the one or more slots may be found elsewhere in the present disclosure (e.g., the description in connection with FIGS. 13A through 16B).

In some embodiments, it may be magnetic conductive between the gantry and the first shielding structure. For example, the gantry may be made of magnetic material (e.g., material with high magnetic susceptibility and permeability, e.g., non-oriented silicon steel). The gantry, the first shielding structure, and a connection component 426 (the gantry may be operably connected to the first shielding structure through the connection component) may form a magnetic circuit. In some embodiments, the gantry and the first shielding structure may be magnetically isolated from each other. For example, the gantry may be made of non-magnetic material.

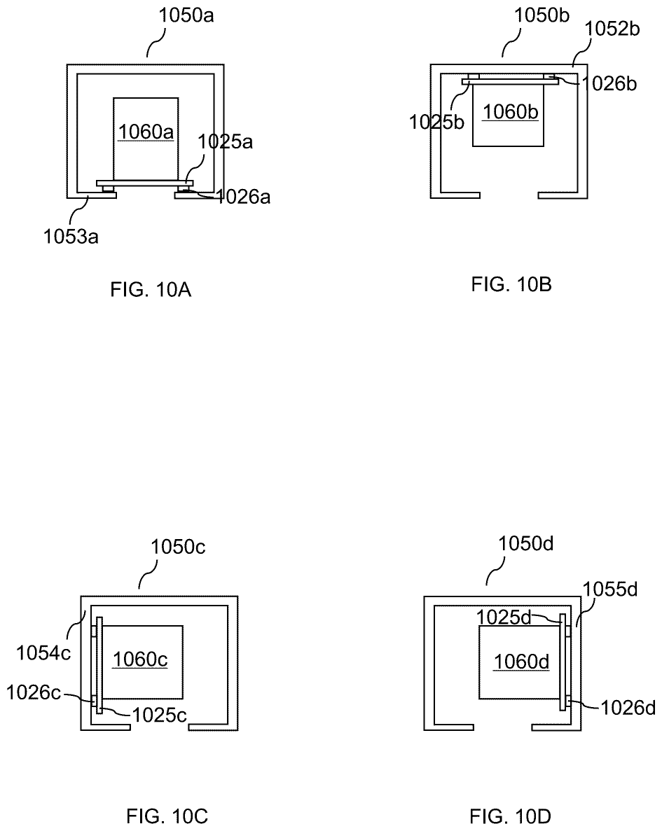

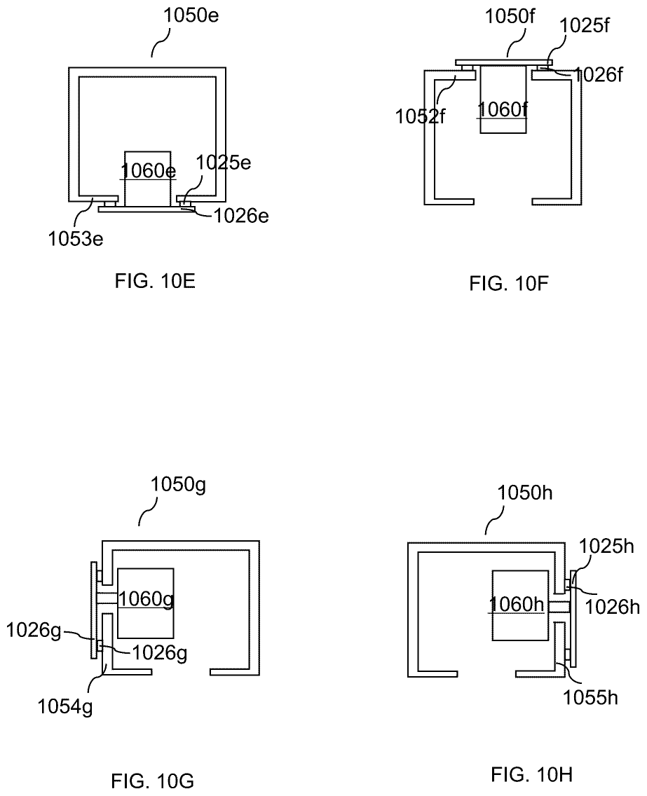

In some embodiments, the radiation therapy device 110 may further include one or more second shielding structures mounted on the gantry and rotatable with the gantry. In some embodiments, two second shielding structures may be respectively disposed at two sides of the linear accelerator of the radiation therapy device 320 along the circumferential direction of the radiation therapy device 110. In some embodiments, a plurality of second shielding structures may be disposed on the gantry along the circumferential direction of the radiation therapy device 110. The one or more second shielding structures may be configured to improve the shielding effect for the MRI device 310 and/or the radiation therapy device 320. Details regarding the configuration of the one or more second shielding structures may be found elsewhere in the present disclosure (e.g., the description in connection with FIGS. 10A through 12).

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. For example, the assembly and/or function of the therapeutic device 110 may vary or change according to a specific implementation scenario. In some embodiments, the radiation therapy device 320 may further include a dose detecting device, a temperature controlling device (e.g., a cooling device), a multiple layer collimator, or the like, or any combination thereof. However, those variations and modifications do not depart from the scope of the present disclosure.

FIG. 4A shows an upper portion of a cross-sectional view of an exemplary therapeutic device 400 viewed along the X direction according to some embodiments of the present disclosure. FIG. 4B shows a perspective view of the therapeutic device 400 according to some embodiments of the present disclosure. As shown in FIG. 4A and FIG. 4B, the X axis, the Y axis, and the Z axis may correspond to those in FIG. 1. In some embodiments, the therapeutic device 110 in FIG. 1 and FIG. 3 may be implemented based on the therapeutic device 400.

As shown in FIG. 4A, the therapeutic device 400 may include an MRI device configured to acquire MRI data with respect to an ROI, a radiation therapy device configured to perform a treatment on at least one portion of the ROI by applying, based on the MRI data, therapeutic radiation to the at least one portion of the ROI, and a first shielding structure 450 configured to provide interference shielding for the MRI device and/or the radiation therapy device. The radiation therapy device may be rotatable around a longitudinal axis 440 (e.g., corresponding to the longitudinal axis 340 of the therapeutic device 110 in FIG. 3) of a bore of the therapeutic device 400.

In some embodiments, the radiation therapy device of the therapeutic device 400 may include a gantry 429 and a radiation source 428 (as shown in FIG. 4B). The radiation source 428 may be configured to emit a radiation beam 427 towards the longitudinal axis 440 (e.g., a treatment region in the bore). The radiation beam 427 may have a central axis 402. The gantry 429 may be configured to support the radiation source 428. The gantry 429, together with the radiation source 428 mounted thereon, may be able to rotate around the longitudinal axis 440. The radiation source 428 may include a linear accelerator 422, a target 423, a collimation component 425, and an MLC 424 that are mounted on the gantry and rotatable around the longitudinal axis 440 with the gantry. In some embodiments, the MLC 424 may be disposed around the longitudinal axis 440. In this case, the MLC 424 may be non-rotatable.

In some embodiments, the MRI device of the therapeutic device 400 may include a magnetic body 411 configured to generate a magnetic field. The magnetic body 411 may be around the longitudinal axis 440. For example, the magnetic body 411 may be an annulus structure around the longitudinal axis 440 with the bore passing through the magnetic body 411 along the extending direction (i.e., the Z direction) of the longitudinal axis 440. In some embodiments, the magnetic body 411 may include a plurality of main magnetic coils and a plurality of shielding magnetic coils arranged coaxially around the longitudinal axis 440. For example, as shown in FIG. 4A, the magnetic body 411 may include three pairs of main magnetic coils (e.g., 408-2) and a pair of shielding magnetic coils (e.g., 408-1). The shielding magnetic coils may be arranged at a larger radius from the longitudinal axis 440 than the main magnetic coils. The main magnetic coils may be configured to generate a magnetic field (e.g., a static magnetic field B0) within a specific region (e.g., a region within the bore of the therapeutic device 400) when the main magnetic coils carry an electric current along a first direction. The shielding magnetic coils may carry an electric current along a second direction that is opposed to the first direction. The shielding magnetic coils may help shield the magnetic field generated by the main magnetic coils on a region outside the MRI device of the therapeutic device 400.

In some embodiments, the magnetic body 411 may include a recess 418 that is around the longitudinal axis 440 and separates the magnetic body 411 into a first chamber 415 and a second chamber 416. For example, the recess 418 may have a shape of an annulus around the longitudinal axis 440.

In some embodiments, the magnetic body 411 may include outer walls 413a-413c and an inner wall 414 coaxially around the longitudinal axis 440. The inner wall 414 may be located closer to the longitudinal axis 440 than the outer walls 413a-413c. The recess 418 may have an opening 401 formed between the outer wall 413a and the outer wall 413b.

As shown in FIG. 4A, the chamber 415 and the chamber 416 may be in fluid communication with each other. The two chambers 415 and 416 may be located at opposite sides of the magnetic body 411 along the extending direction of the longitudinal axis 440 (i.e., the Z direction) and may be connected by a neck chamber 417 between the two chambers 415 and 416. The neck chamber 417 may also be a part of the magnetic body 411 and have a smaller radial size than the two chambers 415 and 416. Each of the chambers 415-417 may have the shape of an annulus with a different outer wall, i.e., the outer walls 413a-413c of the magnetic body 411, respectively. The two chambers 415 and 416 and the neck chamber 417 may share a same inner wall, i.e., the inner wall 414 of the magnetic body 411. The two chambers 415 and 416 may be in fluid communication with each other through the neck chamber 417 between them.

As shown in FIG. 4A, the outer wall 413c of the neck chamber 417 may form an innermost boundary of the recess 418. The recess 418 may have a depth (i.e., the thickness of the annulus in the radial direction) which is defined as the distance from the opening 401 to the outer wall 413c of the neck chamber 417 in the radial direction. In some embodiments, the radiation beam 427 may pass through the neck chamber 417 and emit toward the longitudinal axis 440.

In some embodiments, the recess 418 may be coaxial with the magnetic body 411 along the Y direction. For example, the recess 418 may be coaxial with the magnetic body 411 with respect to the axis 402 that is the central axis of the radiation beam 427. In this case, the recess 418 may separate the magnetic body 411 into two chambers 415 and 416 with a same size.

As shown in FIG. 4A and FIG. 4B, at least a portion of the radiation source 428 may be located within the recess 418. For example, as shown in FIG. 4A, the target 423, the collimation component 425, and the MLC may be completely located within the recess 418. A portion of the linear accelerator 422 may be located within the recess 418, and the rest of the linear accelerator 422 may stretch out of the recess 418 from the opening 401.

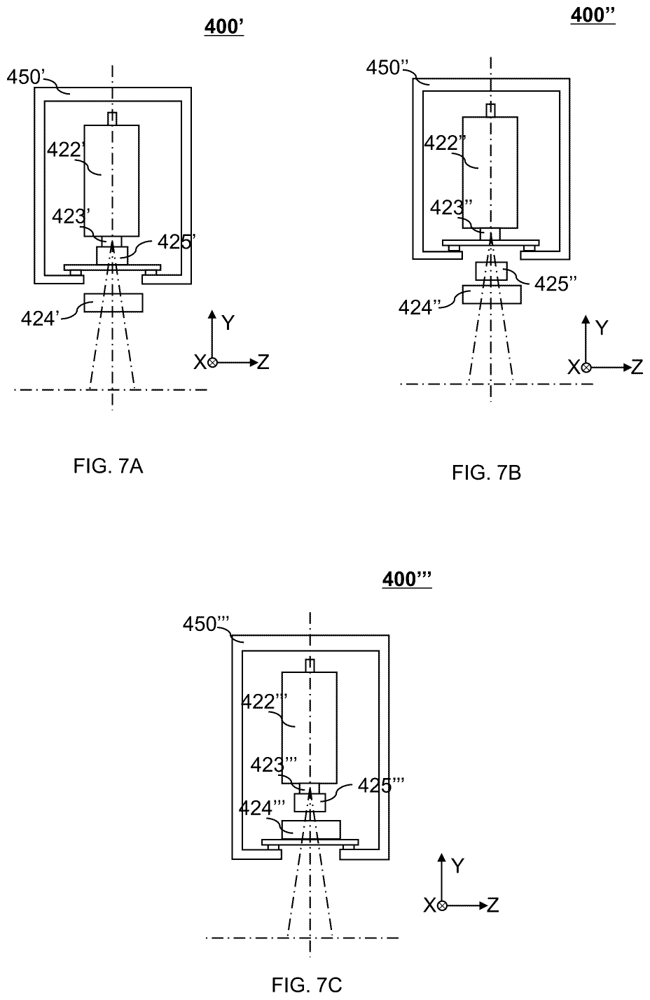

As shown in FIG. 4A, during the treatment, the radiation therapy device may rotate relative to the first shielding structure around the longitudinal axis. Further, during the treatment, the first shielding structure may be non-rotatable around the longitudinal axis (e.g., keep static relative to the MRI device). The first shielding structure 450 may be around the longitudinal axis 440. For example, the first shielding structure 450 may have a shape of an annulus around the longitudinal axis 440. In some embodiments, the radiation source 428 may be at least partially surrounded by the first shielding structure 450. Specifically, at least one of the linear accelerator 422, the target 423, the collimation component 425, or the MLC 424 may be at least partially surrounded by the first shielding structure 450 (as shown in FIGS. 4A and 7A through 7C). For example, as shown in FIG. 4A, the linear accelerator 422 may be located within the first shielding structure 450, and the target 423, the collimation component 425, and the MLC 424 may be located outside the first shielding structure 450.

In some embodiments, the first shielding structure 450 may include four sides, such as an outer side 452 and an inner side 453 along the radial direction, and a front side 455 and a back side 454 disposed opposite to each other along the Z direction. The inner side 453 may be located closer to the longitudinal axis 440 than the outer side 452. In some embodiments, the first shielding structure 450 may include a first opening 451 configured to allow the therapeutic radiation 427 to pass through, so that the therapeutic radiation 427 is able to emit toward the longitudinal axis 440. In some embodiments, the first opening 451 may be disposed on the inner side 453 of the first shielding structure 450. In some embodiments, the smaller the first opening is, the better the shielding effect of the first shielding structure 450 may be.

In some embodiments, the outer side 452 or the inner side 453 may be omitted. For example, the outer side 452 may be omitted. In this case, with the first opening 451 on the inner side 453, the first shielding structure 450 may be regarded as being formed by two L-shaped structures disposed opposite. The inner side 453, the gantry 429, and a connection component 426 (the gantry 429 may be operably connected to the first shielding structure 450 through the connection component 426) may form a magnetic circuit.

In some embodiments, the cross-section of the first shielding structure along the radial direction may have any shape, such as a circle, a rectangle, a square, etc.

In some embodiments, the radiation source 428 (e.g., the linear accelerator 422, the target 423, the collimation component 425, and the MLC 424) may be mounted on the gantry 429 and rotate with the gantry 429. In some embodiments, the gantry 429 may be operably connected to the first shielding structure 450 through a connection component 426. The gantry 429 may be rotatable around the longitudinal axis 440 relative to the first shielding structure 450 through the connection component 426 during the treatment.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 5A shows an upper portion of a cross-sectional view of an exemplary therapeutic device 500-1 viewed along the X direction according to some embodiments of the present disclosure. As shown in FIG. 5A, the X axis, the Y axis, and the Z axis may correspond to those in FIG. 1. In some embodiments, the therapeutic device 110 in FIG. 1 and FIG. 3 may be implemented based on the therapeutic device 500-1. Compared with the therapeutic device 400 described in FIG. 4A, the entire radiation source may be located within the recess 518. Specifically, the linear accelerator 522, the target 523, the collimation component 525, and the MLC 524 may be completely located within the recess 518.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 5B shows an upper portion of a cross-sectional view of an exemplary magnetic body 500-2 viewed along the X direction according to some embodiments of the present disclosure. As shown in FIG. 5B, the X axis, the Y axis, and the Z axis may correspond to those in FIG. 1. In some embodiments, the magnetic body 311 of the therapeutic device 110 in FIG. 3 may be implemented based on the magnetic body 500-2.

Compared with the magnetic body 411 described in FIG. 4A, the magnetic body 500-2 may include a recess 618 that separates the magnetic body 500-2 into two isolated chambers 615 and 616. As shown in FIG. 5B, the chamber 615 and the chamber 616 may be isolated from each other and thus no fluid communication is established between them. The magnetic body 500-2 may include two different inner walls 614a and 614b for the chamber 615 and the chamber 616, respectively. The magnetic body 500-2 may include two different outer walls 613a and 613c for the chamber 615 and the chamber 616, respectively. The recess 618 may include an opening 601 formed between the two different outer walls 613a and 613c, and an opening 603 formed between the two different inner walls 614a and 614b. The radiation beam may pass through the recess 618 from the opening 601 to the opening 603 and emit toward the longitudinal axis 640 (e.g., corresponding to the longitudinal axis 340 in FIG. 3). The recess 418 may have a depth which is defined as the distance from the opening 601 to the opening 602 in the radial direction.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 5C shows an upper portion of a cross-sectional view of an exemplary magnetic body 500-3 viewed along the X direction according to some embodiments of the present disclosure. As shown in FIG. 5C, the X axis, the Y axis, and the Z axis may correspond to those in FIG. 1. In some embodiments, the magnetic body 311 of the therapeutic device 110 in FIG. 3 may be implemented based on the magnetic body 500-3.

Compared with the magnetic body 411 described in FIG. 4A, the magnetic body 500-3 may include a recess 718 that is not coaxial with the magnetic body 500-3 along the Y direction. In this case, the recess 718 may separate the magnetic body 500-3 into two chambers 715 and 716 with different sizes. As shown in FIG. 5C, in the Z direction, the recess 718 is symmetrical with respect to the axis 702 (may also be the central axis of the radiation beam), while the magnetic body 500-3 is symmetrical with respect to the axis 704.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 6 shows an upper portion of a cross-sectional view of an exemplary therapeutic device 600 viewed along the X direction according to some embodiments of the present disclosure. As shown in FIG. 6, the X axis, the Y axis, and the Z axis may correspond to those in FIG. 1. In some embodiments, the therapeutic device 110 in FIG. 1 and FIG. 3 may be implemented based on the therapeutic device 600. Compared with the therapeutic device 400 described in FIG. 4A, there is no recess at the outer wall of the magnetic body 680. The radiation therapy device 670 may be disposed around the outer circumference of the magnetic body 680.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 7A through 7C show upper portions of cross-sectional views of exemplary configurations 400′-400′″ between a first shielding structure and a radiation source viewed along the X direction according to some embodiments of the present disclosure. As shown in FIGS. 7A through 7C, the X axis, the Y axis, and the Z axis may correspond to those in FIG. 1. In some embodiments, the therapeutic device 110 in FIG. 1 and FIG. 3 may be implemented based on the configurations 400′-400′″ illustrated in FIGS. 7A through 7C.

Compared with the therapeutic device 400 described in FIG. 4A, in the configuration 400′, the linear accelerator 422′, the target 423′, and the collimation component 425′ may be located within the first shielding structure 450′, and the MLC 424′ may be located outside the first shielding structure 450′.

Compared with the therapeutic device 400 described in FIG. 4A, in the configuration 400″, the linear accelerator 422″ and the target 423″ may be located within the first shielding structure 450″, and the MLC 424″ and the collimation component 425″ may be located outside the first shielding structure 450″.

Compared with the therapeutic device 400 described in FIG. 4A, in the configuration 400′″, the linear accelerator 422′″, the target 423′″, the MLC 424′″, and the collimation component 425′″ may be located within the first shielding structure 450′″.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIGS. 8A through 9D show an upper portion of a cross-sectional view of configurations 800-1 through 900-4 of a connection between a gantry and a first shielding structure viewed along the X direction according to some embodiments of the present disclosure. As shown in FIGS. 8A through 9D, the X axis, the Y axis, and the Z axis may correspond to those in FIG. 1. In some embodiments, the therapeutic device 110 in FIG. 3 may be implemented based on the configurations 800-1 through 900-4.

In some embodiments, the gantry may be located inside or outside the first shielding structure. In some embodiments, the gantry may be operably connected with any side of the first shielding structure. In some embodiments, when the gantry is located outside the first shielding structure, the first shielding structure may include a second opening configured to facilitate the connection between the gantry and the first shielding structure. The second opening may be disposed on a side of the first shielding structure to which the gantry is operably connected.

As shown in FIG. 8A, in the configuration 800-1, the first shielding structure 850a may include four sides, such as an outer side 852a and an inner side 853a along the radial direction, and a front side 855a and a back side 854a disposed opposite to each other along the Z direction. In some embodiments, the first shielding structure 850a may include a first opening 851a configured to allow the therapeutic radiation 827a to pass through, so that the therapeutic radiation 827a emitting from the radiation source 828a is able to emit toward the longitudinal axis 840a (e.g., corresponding to the longitudinal axis 340 in FIG. 3). In some embodiments, the first opening 851a may be disposed on the inner side 853a of the first shielding structure 850a.

As shown in FIG. 8A, the gantry 825a may be located inside the first shielding structure 850a. The gantry 825a may be operably connected to the inner surface of the inner side 853a of the first shielding structure 850a through the connection component 826a. The gantry 825a may be rotatable around the longitudinal axis 840a within and relative to the first shielding structure 850a through the connection component 825a during a treatment.

Compared to the configuration 800-1 in FIG. 8A, in the configuration 800-2 in FIG. 8B, the gantry 825b may be operably connected to the inner surface of the outer side 852b of the first shielding structure 850b through the connection component 826b.

Compared to the configuration 800-1 in FIG. 8A, in the configuration 800-3 in FIG. 8C, the gantry 825c may be operably connected to the inner surface of the back side 854c of the first shielding structure 850c through the connection component 826c.

Compared to the configuration 800-1 in FIG. 8A, in the configuration 800-4 in FIG. 8D, the gantry 825d may be operably connected to the inner surface of the front side 855d of the first shielding structure 850d through the connection component 826d.

Compared to the configuration 800-1 in FIG. 8A, in the configuration 900-1 in FIG. 9A, the gantry 925a may be located outside the first shielding structure 950a. The gantry 925a may be operably connected to the outer surface of the inner side 953a of the first shielding structure 950a through the connection component 926a. The first opening 951a may be configured to not only allow the therapeutic radiation 927a to pass through, so that the therapeutic radiation 927a emitting from the radiation source 928a is able to emit toward the longitudinal axis 940a (e.g., corresponding to the longitudinal axis 340 in FIG. 3), but also facilitate the connection between the gantry 925a and the first shielding structure 950a.

Compared to the configuration 800-2 in FIG. 8B, in the configuration 900-2 in FIG. 9B, the gantry 925b may be located outside the first shielding structure 950b. The gantry 925b may be operably connected to the outer surface of the outer side 952b of the first shielding structure 950b through the connection component 926b. The first shielding structure 950b may further include a second opening 956b disposed on the outer side 952b of the first shielding structure 950b and configured to facilitate the connection between the gantry 925b and the first shielding structure 950b.

Compared to the configuration 800-3 in FIG. 8C, in the configuration 900-3 in FIG. 9C, the gantry 925c may be located outside the first shielding structure 950c. The gantry 925c may be operably connected to the outer surface of the back side 954c of the first shielding structure 950c through the connection component 926c. The first shielding structure 950c may further include a second opening 956c disposed on the back side 954c of the first shielding structure 950c and configured to facilitate the connection between the gantry 925c and the first shielding structure 950c.

Compared to the configuration 800-4 in FIG. 8D, in the configuration 900-4 in FIG. 9D, the gantry 925d may be located outside the first shielding structure 950d. The gantry 925d may be operably connected to the outer surface of the outer side 955d of the first shielding structure 950d through the connection component 926d. The first shielding structure 950d may further include a second opening 956d disposed on the front side 955d of the first shielding structure 950d and configured to facilitate the connection between the gantry 925d and the first shielding structure 950d.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, the therapeutic device 110 may further include one or more second shielding structures mounted on the gantry and inside the first shielding structure (e.g., as shown in FIGS. 10A through 10H).

For example, FIG. 10A shows a cross-sectional view of an exemplary second shielding structure 1060a mounted on a circumferential position of the gantry 1025a along the radial direction according to some embodiments of the present disclosure. As shown in FIG. 10A, the second shielding structure 1060a and the gantry 1025a may be located inside the first shielding structure 1050a. The gantry 1025a may be operably connected to the inner surface of the lower side 1053a of the first shielding structure 1050a through the connection component 1026a.

As another example, FIG. 10B shows a cross-sectional view of an exemplary second shielding structure 1060b mounted on a circumferential position of the gantry 1025b along the radial direction according to some embodiments of the present disclosure. As shown in FIG. 10B, the second shielding structure 1060b and the gantry 1025b may be located inside the first shielding structure 1050b. The gantry 1025b may be operably connected to the inner surface of the upper side 1052b of the first shielding structure 1050b through the connection component 1026b.

As still another example, FIG. 10C shows a cross-sectional view of an exemplary second shielding structure 1060c mounted on a circumferential position of the gantry 1025c along the radial direction according to some embodiments of the present disclosure. As shown in FIG. 10C, the second shielding structure 1060c and the gantry 1025c may be located inside the first shielding structure 1050c. The gantry 1025c may be operably connected to the inner surface of the back side 1054c of the first shielding structure 1050c through the connection component 1026c.

As still another example, FIG. 10D shows a cross-sectional view of an exemplary second shielding structure 1060d mounted on a circumferential position of the gantry 1025d along the radial direction according to some embodiments of the present disclosure. As shown in FIG. 10D, the second shielding structure 1060d and the gantry 1025d may be located inside the first shielding structure 1050d. The gantry 1025d may be operably connected to the inner surface of the front side 1055d of the first shielding structure 1050d through the connection component 1026d.

As still another example, FIG. 10E shows a cross-sectional view of an exemplary second shielding structure 1060e mounted on a circumferential position of the gantry 1025e along the radial direction according to some embodiments of the present disclosure. As shown in FIG. 10E, the second shielding structure 1060e may be located inside the first shielding structure 1050e. The gantry 1025e may be located outside the first shielding structure 1050e. The gantry 1025e may be operably connected to the outer surface of the lower side 1053e of the first shielding structure 1050e through the connection component 1026e.

As still another example, FIG. 10F shows a cross-sectional view of an exemplary second shielding structure 1060f mounted on a circumferential position of the gantry 1025f along the radial direction according to some embodiments of the present disclosure. As shown in FIG. 10F, the second shielding structure 1060f may be located inside the first shielding structure 1050f. The gantry 1025f may be located outside the first shielding structure 1050f. The gantry 1025f may be operably connected to the outer surface of the upper side 1052f of the first shielding structure 1050f through the connection component 1026f.

As still another example, FIG. 10G shows a cross-sectional view of an exemplary second shielding structure 1060f mounted on a circumferential position of the gantry 1025f along the radial direction according to some embodiments of the present disclosure. As shown in FIG. 10G, the second shielding structure 1060g may be located inside the first shielding structure 1050g. The gantry 1025g may be located outside the first shielding structure 1050g. The gantry 1025g may be operably connected to the outer surface of the back side 1054g of the first shielding structure 1050g through the connection component 1026g.

As still another example, FIG. 10H shows a cross-sectional view of an exemplary second shielding structure 1060h mounted on a circumferential position of the gantry 1025h along the radial direction according to some embodiments of the present disclosure. As shown in FIG. 10H, the second shielding structure 1060h may be located inside the first shielding structure 1050h. The gantry 1025h may be located outside the first shielding structure 1050h. The gantry 1025h may be operably connected to the outer surface of the front side 1055h of the first shielding structure 1050h through the connection component 1026h.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

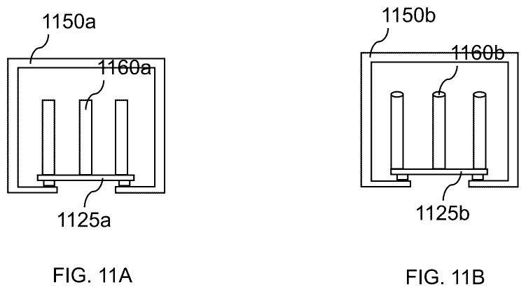

In some embodiments, the second shielding structure may have a shape of a plate (e.g., as shown in FIGS. 10A through 10H). In some embodiments, the second shielding structure may include one or more parallel strips or rods (e.g., as shown in FIGS. 11A and 11B). It shall be noted that the number of the strips or rods at each circumferential position on the gantry can be any suitable integer, such as, 1, 2, 3, 4, etc.

For example, FIG. 11A shows a cross-sectional view of an exemplary second shielding structure 1160a at a circumferential position on the gantry along the radial direction according to some embodiments of the present disclosure. As shown in FIG. 11A, the second shielding structure 1160a may include 3 parallel strips mounted on the gantry 1125a. The second shielding structure 1100-1 may be located inside the first shielding structure 1150a.

As another example, FIG. 11B shows a cross-sectional view of an exemplary second shielding structure 1160b at a circumferential position on the gantry along the radial direction according to some embodiments of the present disclosure. As shown in FIG. 11B, the second shielding structure 1160b may include 3 parallel rods mounted on the gantry 1125b. The second shielding structure 1160b may be located inside the first shielding structure 1150b.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

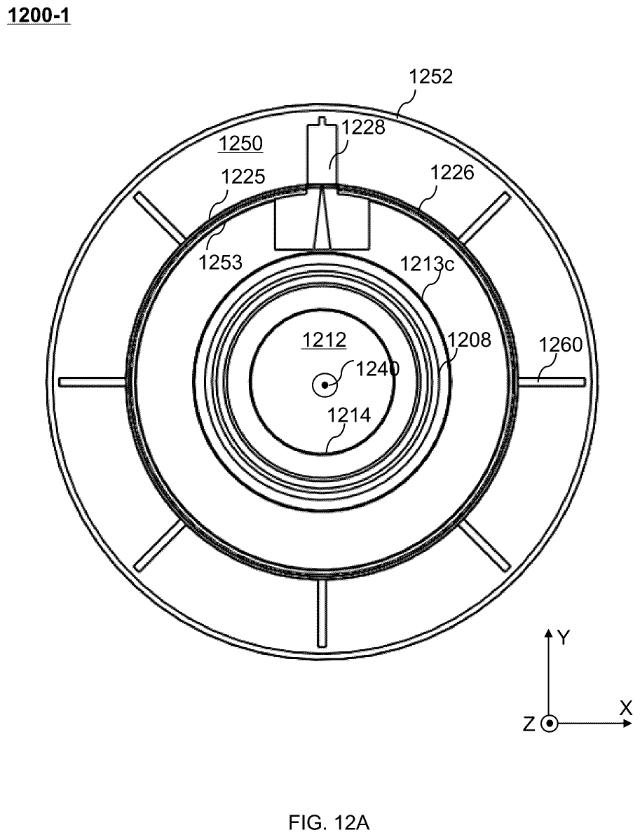

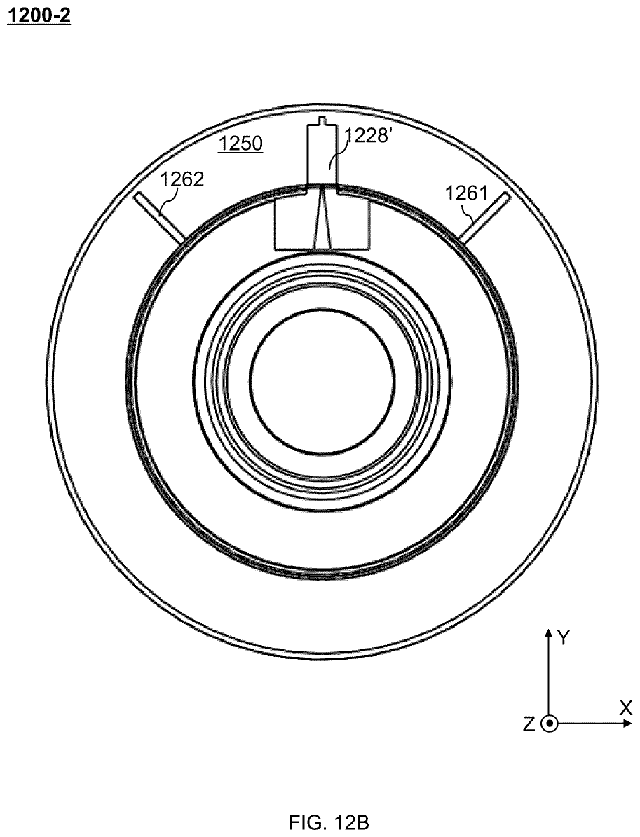

In some embodiments, the one or more second shielding structures may be respectively located at one or more circumferential locations on the gantry. In some embodiments, the radiation source and a plurality of second shielding structures may be evenly distributed on the gantry (e.g., as shown in FIG. 12A). In some embodiments, two second shielding structures may be respectively disposed at two sides of the radiation source along the circumferential direction (e.g., as shown in FIG. 12B).

For example, FIG. 12A shows a cross-sectional view of an exemplary therapeutic device 1200-1 viewed along the Z direction according to some embodiments of the present disclosure. As shown in FIG. 12A, the X axis, the Y axis, and the Z axis may correspond to those in FIG. 1. In some embodiments, the therapeutic device 110 in FIG. 1 and FIG. 3 may be implemented based on the therapeutic device 1200-1.

As shown in FIG. 12A, in the therapeutic device 1200-1, the first shielding structure 1250 may include an outer side 1252 and an inner side 1253 coaxially around the longitudinal axis 1240 (e.g., corresponding to the longitudinal axis 340 in FIG. 3). The radiation source 1228 may be mounted on the gantry 1225. The gantry 1225 and the radiation source 1228 may be located inside the first shielding structure 1250. The gantry 1225 may be operably connected to the inner surface of the inner side 1253 through the connection component 1226. The therapeutic device 1200-1 may include an MRI device including a magnetic body that includes an inner wall 1214. The inner wall 1214 may define a bore 1212 of the MRI device. The magnetic body may include a plurality of coils 1208. The MRI device of the therapeutic device 1200-1 may include a recess that separates the magnetic body into two chambers in fluid communication with each other through a neck chamber. The recess may have an innermost boundary 1213c. The innermost boundary 1213c may also be the outer wall of the neck chamber. The innermost boundary 1213c and the inner wall 1214 of the magnetic body may define the neck chamber.

As shown in FIG. 12A, the therapeutic device 1200-1 may include a plurality of second shielding structures 1260 respectively located at a plurality of circumferential locations on the gantry 1225. As shown in FIG. 12A, the radiation source 1228 and the second shielding structures 1260 may be evenly distributed on the gantry 1225.

As another example, FIG. 12B shows a cross-sectional view of an exemplary therapeutic device 1200-1 viewed along the Z direction according to some embodiments of the present disclosure. As shown in FIG. 12B, the X axis, the Y axis, and the Z axis may correspond to those in FIG. 1. In some embodiments, the therapeutic device 110 in FIG. 1 and FIG. 3 may be implemented based on the therapeutic device 1200-2.

As shown in FIG. 12A, compared with the therapeutic device 1200-1, the therapeutic device 1200-2 may include two second shielding structures 1261 and 1262 respectively located at two sides of the radiation source 1228′ along the circumferential direction. In some embodiments, the two second shielding structures 1261 and 1262 may be symmetrical with respect to the radiation source 1228′.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, the first shielding structure may include one or more slots configured to dissipate heat produced by the MRI device or the radiation therapy device, or facilitate cable layout of the therapeutic device 110. In some embodiments, the one or more slots may be disposed on at least one of the four sides of the first shielding structure. In some embodiments, the distance between any two neighboring slots disposed on the same side of the first shielding structure may be the same or different. In some embodiments, the distance between any two neighboring slots disposed on different sides of the first shielding structure may be the same or different. Each slot may have a shape of a rectangle, an ellipse, or the like. In some embodiments, a slot disposed on the outer side or the inner side of the first shielding structure may extend along the Z direction. A slot disposed on the back side or the front side of the first shielding structure may extend along a corresponding radial direction. In some embodiments, a slot disposed on a side of the first shielding structure may not penetrate the side of the first shielding structure along the slot's extending direction. In some embodiments, a slot disposed on a side of the first shielding structure may penetrate the side of the first shielding structure along the slot's extending direction. In this way, the side of the first shielding structure may be separated into a plurality of discrete portions by one or more penetrating slots that are disposed on the side of the first shielding structure.

For example, as shown in FIG. 13A, a slot 1357a may be disposed on the back side 1354a of the first shielding structure 1350a. The slot 1357a may extend along the radial direction corresponding to the slot 1357a and may not penetrate the back side 1354a along the corresponding radial direction.

As another example, as shown in FIG. 13B, a slot 1357b may be disposed on the front side 1355b of the first shielding structure 1350b. The slot 1357b may extend along the radial direction corresponding to the slot 1357b and may not penetrate the front side 1355b along the corresponding radial direction.

As still another example, as shown in FIG. 13C, a slot 1357c may be disposed on the outer side 1352c of the first shielding structure 1350c. The slot 1357c may extend along the Z direction and may not penetrate the outer side 1352c along the Z direction.

As still another example, as shown in FIG. 13D, a slot 1357d may be disposed on the inner side 1353d of the first shielding structure 1350d. The slot 1357d may extend along the Z direction and may not penetrate the inner side 1353d along the Z direction. The slot 1357d may intersect with the first opening 1351d so as to be separated into portion 1357d-1 and portion 1357d-2 by the first opening 1351d.

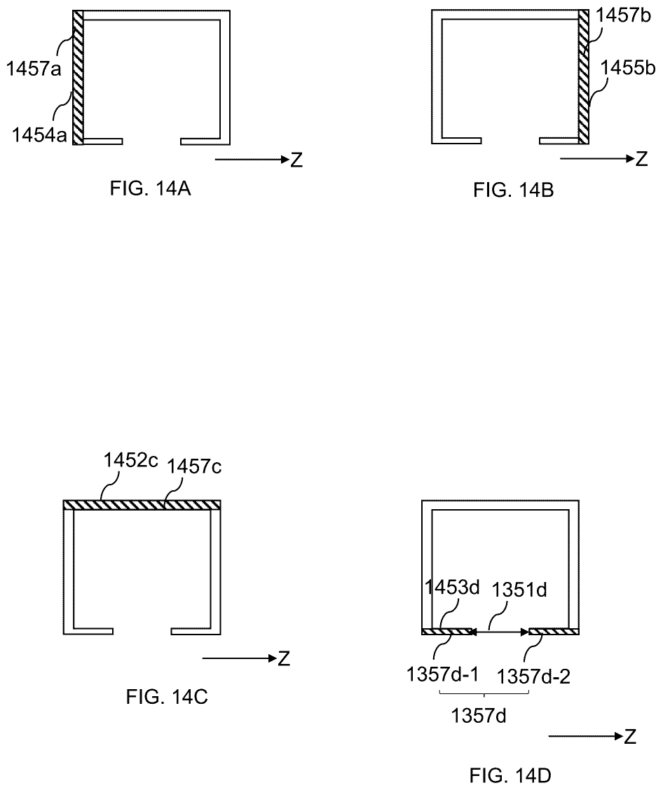

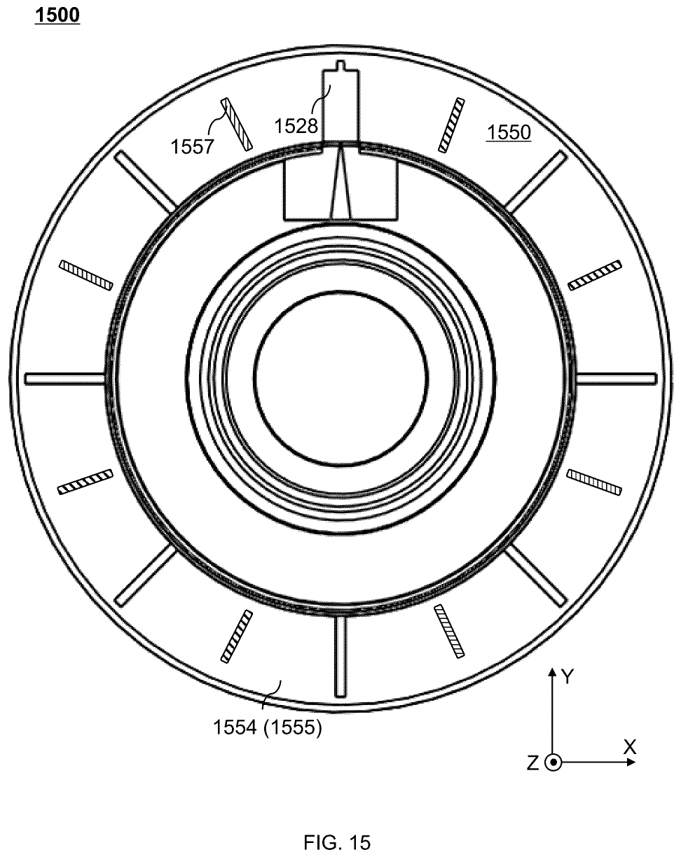

As still another example, as shown in FIG. 14A, a slot 1457a may be disposed on the back side 1454a of the first shielding structure 1350a. The slot 1457a may extend along the radial direction corresponding to the slot 1457a and may penetrate the back side 1454a along the corresponding radial direction.

As still another example, as shown in FIG. 14B, a slot 1457b may be disposed on the front side 1455b of the first shielding structure 1450b. The slot 1457b may extend along the radial direction corresponding to the slot 1457b and may penetrate the front side 1454b along the corresponding radial direction.

As still another example, as shown in FIG. 14C, a slot 1457c may be disposed on the outer side 1452c of the first shielding structure 1450c. The slot 1457c may extend along the Z direction and may penetrate the outer side 1452c along the Z direction.

As still another example, as shown in FIG. 14D, a slot 1457d may be disposed on the inner side 1453d of the first shielding structure 1450d. The slot 1457d may extend along the Z direction and may penetrate the inner side 1453d along the Z direction. The slot 1457d may intersect with the first opening 1451d so as to be separated into portion 1457d-1 and portion 1457d-2 by the first opening 1451d.

As shown in FIGS. 13A through 14D, the Z axis may correspond to the Z axis in FIG. 1.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, a slot may be disposed between the radiation source and a second shielding structure, or between two adjacent second shielding structures.

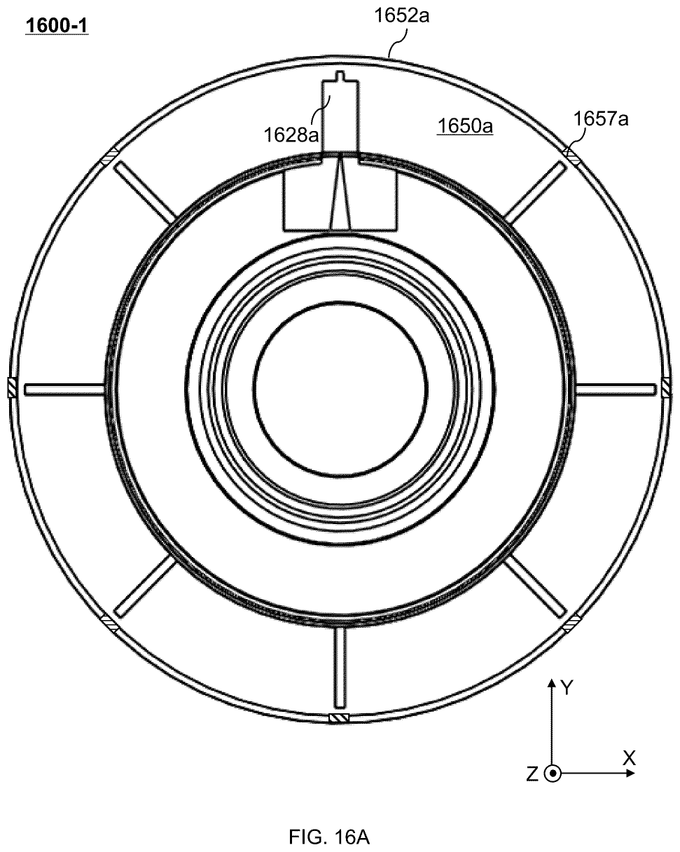

FIG. 15 shows a cross-sectional view of an exemplary therapeutic device 1500 viewed along the Z direction according to some embodiments of the present disclosure. As shown in FIG. 15, the X axis, the Y axis, and the Z axis may correspond to those in FIG. 1. In some embodiments, the therapeutic device 110 in FIG. 3 may be implemented based on the therapeutic device 1500.

Compared to the therapeutic device 1200-1 illustrated in FIG. 12A, as shown in FIG. 15, a plurality of slots 1557 may be disposed on the back side 1554 (and/or the front side 1555) of the first shielding structure 1550. Each of the plurality of slots 1557 may extend along a corresponding radial direction and not penetrate the back side 1554 (and/or the front side 1555) along the corresponding radial direction. In some embodiments, as shown in FIG. 15, when the radiation source 1528 is located at an initial location, each of the plurality of slots 1557 may be located between the radiation source 1528 and the second shielding structure, or between two adjacent second shielding structures. For example, the radiation source 1528 may be rotatable around the Z axis. Among the locations along the circumferential direction around the Z axis, the location at the positive Y direction along the Y axis may be referred to as the initial location.

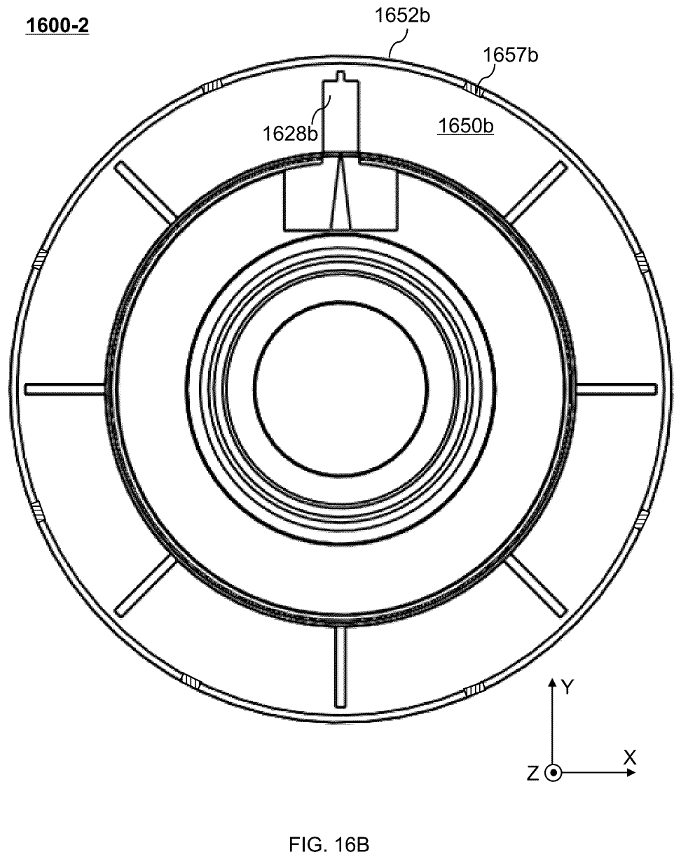

FIG. 16A shows a cross-sectional view of an exemplary therapeutic device 1600-1 viewed along the Z direction according to some embodiments of the present disclosure. As shown in FIG. 16A, the X axis, the Y axis, and the Z axis may correspond to those in FIG. 1. In some embodiments, the therapeutic device 110 in FIG. 3 may be implemented based on the therapeutic device 1600-1.

Compared to the therapeutic device 1500 illustrated in FIG. 15, as shown in FIG. 16A, a plurality of slots 1657a may be disposed on the outer side 1652a of the first shielding structure 1650a. Each of the plurality of slots 1657a may extend along the Z direction. In some embodiments, as shown in FIG. 16A, when the radiation source 1628a is located at the initial location, each of the plurality of slots 1657a may be located corresponding to one of the second shielding structures.

FIG. 16B shows a cross-sectional view of an exemplary therapeutic device 1600-2 viewed along the Z direction according to some embodiments of the present disclosure. As shown in FIG. 16B, the X axis, the Y axis, and the Z axis may correspond to those in FIG. 1. In some embodiments, the therapeutic device 110 in FIG. 3 may be implemented based on the therapeutic device 1600-2.

Compared to the therapeutic device 1600-2 illustrated in FIG. 16A, as shown in FIG. 16B, when the radiation source 1628b is located at the initial location, each of a plurality of slots 1657a disposed on the outer side 1652b of the first shielding structure 1650b may be located between the radiation source 1628b and the second shielding structure, or between two adjacent second shielding structures.

It should be noted that the above description of the therapeutic device 110 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A radiation therapy system comprising:
a magnetic resonance imaging (MRI) device configured to acquire MRI data with respect to a region of interest (ROI), the MRI device including:
a main magnet that is around a longitudinal axis and configured to generate a magnetic field;
a radiation therapy device configured to perform a treatment on at least one portion of the ROI by delivering, based on the MRI data, therapeutic radiation to the at least one portion of the ROI, the radiation therapy device being rotatable around the longitudinal axis; and
a first shielding structure configured to provide interference shielding for the MRI device or the radiation therapy device, the radiation therapy device being rotatable relative to the first shielding structure around the longitudinal axis.

2. The radiation therapy system of claim 1, wherein the first shielding structure is around the longitudinal axis.

3. The radiation therapy system of claim 1, wherein the radiation therapy device is at least partially surrounded by the first shielding structure.

4. The radiation therapy system of claim 3, wherein the first shielding structure includes a first opening configured to allow the therapeutic radiation from the radiation therapy device to pass through.

5. The radiation therapy system of claim 1, wherein the radiation therapy device further includes:
a radiation source configured to provide the therapeutic radiation; and
a gantry configured to support the radiation source, the radiation source being rotatable with the gantry.

6. The radiation therapy system of claim 5, wherein the radiation therapy device further includes:
a connection component configured to operably connect the gantry and the first shielding structure, the gantry being rotatable around the longitudinal axis and supported on the first shielding structure through the connection component.

7. The radiation therapy system of claim 6, wherein the connection component includes one or more bearings.

8. The radiation therapy system of claim 5, further comprising:
one or more second shielding structures mounted on the gantry.

9. The radiation therapy system of claim 8, wherein the one or more second shielding structures are respectively located at one or more circumferential locations on the gantry.

10. The radiation therapy system of claim 9, wherein the radiation source and the one or more second shielding structures are evenly distributed on the gantry.

11. The radiation therapy system of claim 5, wherein the gantry is located within the first shielding structure.

12. The radiation therapy system of claim 5, wherein the gantry is located outside the first shielding structure.

13. The radiation therapy system of claim 1, wherein there is a recess at an outer wall of the main magnet, the recess separating the main magnet into two chambers.

14. The radiation therapy system of claim 13, wherein the radiation therapy device is at least partially located within the recess.

15. The radiation therapy system of claim 13, wherein the two chambers are in fluid communication with each other.

16. The radiation therapy system of claim 15, wherein the two chambers are connected through a neck chamber, the recess being at least defined by the two chambers and the neck chamber.

17. The radiation therapy system of claim 13, wherein the two chambers are isolated from each other.

18. The radiation therapy system of claim 1, wherein the radiation therapy device further includes at least one of:
a linear accelerator configured to accelerate electrons in an electron beam to produce a radiation beam of the therapeutic radiation,
a target configured to receive the accelerated electron beam to produce the radiation beam for the therapeutic radiation,
a collimation component configured to collimate the radiation beam of the therapeutic radiation, or
a multi-leaf collimator (MLC) configured to make the radiation beam approximate the at least one portion of the ROI;
wherein at least one of the linear accelerator, the target, the collimation component, or the MLC is at least partially surrounded by the first shielding structure.

19. (canceled)

20. The radiation therapy system of claim 1, wherein the first shielding structure includes one or more slots configured to dissipate heat produced by the MRI device or the radiation therapy device, or facilitate cable layout of the radiation therapy system.

21. The radiation therapy system of claim 1, wherein the first shielding structure is non-rotatable around the longitudinal axis during the treatment.
